(19) United States
(12) Patent Application Publication
(10) Pub. No.: US 2019/0338280 A1
(43) Pub. Date: Nov. 7, 2019

(54) IMMUNOMODULATION BY CONTROLLING ELR+ PROINFLAMMATORY CHEMOKINE LEVELS WITH THE LONG NON-CODING RNA UMLILO

(71) Applicant: CSIR, Pretoria (ZA)

(72) Inventors: Emiliano DALLA, Trieste (IT); Musa M. MHLANGA, Johannesburg (ZA); Stephanie FANUCCHI, Johannesburg (ZA); Youtaro SHIBAYAMA, Pretoria (ZA)

(21) Appl. No.: 16/220,270

(22) Filed: Dec. 14, 2018

Related U.S. Application Data
(63) Continuation of application No. 15/537,362, filed on Jun. 16, 2017, now abandoned.

(30) Foreign Application Priority Data
Dec. 18, 2014 (ZA) 2014/09351

(51) Int. Cl. C12N 15/113; C12N 7/00

(52) U.S. Cl. CPC C12N 15/113; C12N 7/00; C12N 2740/15041; C12N 2310/14; C12N 2310/113; C12N 2310/531; C12N 2310/20; C12N 2310/17

(57) ABSTRACT

The present invention relates to methods for modulating the production of ELR+ proinflammatory chemokines in a subject or a cell using either UMLILO IncRNA inhibitors to decrease production of ELR+ proinflammatory cytokines or using UMLILO IncRNA's to increase the production of ELR+ proinflammatory cytokines. The invention also provides for the use of UMLILO IncRNA inhibitors or UMLILO IncRNA's to modulate the expression of ERL+ proinflammatory cytokines.

### CROSS-REFERENCE TO RELATED APPLICATIONS

This application is a Continuation of U.S. application Ser. No. 15/537,362, filed 16 Jun. 2017, which is a National Stage entry of International Application No. PCT/IB2015/059783, filed Dec. 18, 2015, which claims priority to South African Patent Application No. 2014/09351, filed Dec. 18, 2014.

### REFERENCE TO SEQUENCE LISTING SUBMITTED AS A COMPLIANT ASCII TEXT FILE (.txt)

Pursuant to the EFS-Web legal framework and 37 CFR §§ 1.821-825 (see MPEP § 2442.03(a)), a Sequence Listing in the form of an ASCII-compliant text file (entitled “Sequence_Listing_3000012-005001_ST25.bd” created on 13 Dec. 2018, and 62,604 bytes in size) is submitted concurrently with the instant application, and the entire contents of the Sequence Listing are incorporated herein by reference.

### BACKGROUND OF THE INVENTION

The present invention pertains generally to immunomodulation by altering levels of the proinflammatory chemokines. In particular, the invention relates to methods of modulating an immune response with UMLILO, a long non-coding RNA that regulates expression of the ELR+ proinflammatory chemokines by binding to WRD5. The invention further relates to methods of treating inflammatory, autoimmune, and infectious diseases, immunodeficiency, and cancer by reducing or increasing levels of chemokines.

Inflammation is a natural defense against pathogens as well as a hallmark of several chronic diseases. Inflammatory signals are conveyed by cytokines, a diverse group of small proteins secreted by immune and non-immune cells. Chemokines, the largest group of cytokines, induce neutrophil chemotaxis to the site of injury or infection. As this response forms a critical component of the innate immune system, it is precisely calibrated according to the nature and strength of the stimuli. Inadequate calibration may preclude clearing of infection, whereas excessive neutrophil chemotaxis will lead to tissue degradation and eventually chronic inflammatory disorders such as cancer, autoimmune disease, rheumatoid arthritis and severe sepsis.

Within the eukaryotic nucleus, the three-dimensional compaction of chromatin is tightly linked to transcriptional activity. The nucleus is an immensely crowded, yet paradoxically, highly organized environment. Its main constituent, DNA, is folded into chromosomal loops at the completion of mitosis, allowing its compaction in 3D nuclear space. Chromosomal folding and looping has been demonstrated to be causal to transcription bringing co-regulated genes and enhancer-promoter elements together in cis and trans, to form multigene complexes. These chromosomal contacts are usually identified by the population-based chromosome conformation capture (3C) and derivative techniques (Hi-C, ChIA-PET, 5C, 4C). Analyses of Hi-C data reveal that chromatin is divided into domains enriched in multigene complexes, referred to as topologically associating domains (TADs), with a median length of ˜180 Kbp. In metazoans, TADs are highly conserved and possess well-defined boundaries that are maintained across cell types. However, as 3C-based data sets are typically averaged over millions of cells, these interaction maps most likely represent an ensemble of dynamic chromosomal contacts, rather than revealing that the structure of TADs are identical in every cell across the population. Indeed, single cell Hi-C studies reveal that individual cells within a homogenous population may each possess different 3D chromatin organization. This highlights the absolute necessity of overlaying global interactome data with single cell studies.

Within TADs, or at the interface between neighbouring TADs, chromosomal looping brings genes and enhancer-promoter elements into close proximity, thereby increasing the concentration of transcriptional regulators in multigene complexes. The stability of these contacts remains an open question, with recent evidence showing that these contacts may be formed prior to gene activation, whereas alternative evidence favours the view that these interactions are dynamically established. Seminal studies of the β-globin locus reveal that the locus control region (LCR) forms dynamic chromosomal interactions with the promoter of the β-globin gene. Interestingly, single cell studies using fluorescent in situ hybridization (FISH) assays reveal that the LCR-β-globin chromosomal interactions are confined to a small subset of the population referred to as “jackpot cells”, which also exhibit ˜100 fold higher levels of β-globin expression. Transcription is an inherently stochastic process, which is regulated by several factors, including the random collision of transcription factors with upstream promoter and enhancer DNA regions. Thus, the enrichment of chromosomal interactions, such as those observed between the LCR and β-globin, may account for the increased heterogeneity, or all-or-nothing transcriptional response, observed in a subset of an otherwise identical population of cells. However, dynamic loop-mediated regulation, and the resulting stochastic responses in gene expression, may not be ideal for gene classes that need to respond both immediately and uniformly to external stimuli across cell populations. This suggests that to reduce stochasticity in gene expression, the chromosomal contact between rapidly responding genes may exist in a more stable, or pre-formed state. This strongly implicates the influence of chromosomal looping and TAD structure on this sub-class of transcriptional responses.

The syntenic arrangement of co-regulated genes into TADs may enhance the probability of genes engaging in loop-mediated contact within multigene complexes. Recently, Hi-C studies revealed that several classes of innate immune genes are segregated into TADs and interact within multigene complexes. Rapid gene activation is critical to mount a successful innate immune response, which is an evolutionarily ancient system that protects the host organism against invading pathogens and other infectious agents. In particular, the rapid and robust transcriptional induction of the ELR+ CXC class of chemokines (IL8, CXCL1, CXCL2 and CXCL3) is necessary to induce neutrophil chemotaxis, the first line of the mammalian innate immune defense. Critically, a detailed understanding of the molecular mechanism underpinning rapid CXC chemokine transcriptional regulation is lacking. 3C analysis revealed that the promoters of several classes of the innate immune genes engage in chromosomal contact prior to activation by TNF. In addition, strongly emphasizing the central role of the transcriptional cycle in shaping the inflammatory response, the promoters of the innate immune response genes are preoccupied with poised RNA Pol II and are marked with active histone marks, such as trimethylation of lysine-4 of histone H3 (H3K4me3). This suggests that pre-formed chromosomal contact may be a prerequisite to poise an innate immune gene for expression, but, that an additional signal is necessary for complete transcriptional activation. As the transition between inactive and active promoter states is typically a slow, this may be a mechanism that eukaryotes have evolved to reduce the intrinsic noise associated with transcription. Uncontrolled CXC chemokine expression is the etiology of many disorders with an inflammatory basis, such as inflammatory bowel disease, cancer, autoimmune disease and severe sepsis. The latter is responsible for the highest patient mortality in the ICU. Therefore, decrypting the contribution of stable, or pre-formed, chromosomal contact to the poised nature of the innate immune genes would contribute significantly to achieving a full grasp of the molecular mechanism underpinning the uniform, rapid and robust activation of CXC chemokine transcription.

Many critical proinflammatory genes, such as the chemokines, exhibit accessible promoters and are therefore primed for rapid activation. The promoters of these genes are enriched for histone H3 K4 trimethylation (H3K4me3), an active chromatin mark that is catalyzed by the MLL family of methyltransferases. This chromatin modification permits RNA Pol II to access the promoter. The release of “paused” Pol II is facilitated by the recruitment of the transcriptional elongation factor p-TEFb by the chromatin reader, BRD4.

MLL1 is one of six members of the MLL family, which regulates hematopoiesis through the regulation of H3K4 methylation of the Hox genes. MLL1 has been implicated as the H3K4 methyltransferase of the innate immunity genes as it has been found to selectively regulate the activation of TNF- and LPS-induced gene activation (Wang et al. 2012). MLL1 activity is mediated via direct protein-protein interactions with the WAR complex (comprised of WDR5, Ash2L and RbBP5) as well as other regulators. Activity of MLL1 is particularly reliant on its association with WDR5, a multifunctional adapter protein that identifies H3K4 methylation marks on the promoters of genes as well as binds to MLL1. Therefore, small molecule inhibitors such as MM-401, MM-102 or OICR-9429 that prevent the WDR5/MLL1 interaction lead to a significant reduction in MLL1 methyltransferase activity (Cao et al. 2014; Griebien et al. 2015).

WDR5 has been shown to bind directly to long non coding RNAs. Long intergenic non coding RNA (lincRNA) transcripts range from ≤200nt to over 10kb in length and may even be spliced and polyadenylated. A subset of IncRNAs has been shown to regulate gene activity by binding to active or repressive chromatin remodeling complexes. Specifically, the IncRNA Xist achieves silencing of the X chromosome and dosage compensation by interacting with the polycomb repressive complex 2 (PRC2) leading to H3K27me3 and gene silencing. In contrast, two IncRNAs have been identified that bind to WDR5 and facilitate epigenetic H3K4me3 activation. HOTTIP is IncRNA of the human HOXA locus that binds directly binds to the WDR5 protein to recruit the MLL H3K4 methylase complex to maintain H3K4me3 (Wang et al. 2011). In addition, recent research has shown that the NeST IncRNA upregulates IFN-γ expression through interactions with WDR5 (Gomez et al. 2013, patent publication number US 2014/0056929 A1).

By acting as intermediates that link nuclear organization to gene regulation, long noncoding RNAs (IncRNAs) are emerging to be key modulators of gene activity. Despite lacking full protein coding potential, IncRNAs have been demonstrated to be essential during development with alterations in IncRNA activity shown to be associated with a multitude of disease states, such as cancer and inflammation. Although thousands of IncRNAs have been identified, very few have been assigned a function. A large portion of the characterised IncRNAs appear to act as a scaffold for chromatin remodeling complexes that read, write (Wang et al. 2011; Gomez et al. 2013) or erase histone modifications. In this way, IncRNAs may act in cis or trans to create local ‘nuclear compartments’ of high IncRNA-protein concentration which are able to modulate the epigenome, and as a consequence, the activity of target genes (Wang et al. 2011; Gomez et al. 2013). This reveals that the appropriate genomic location of IncRNAs is critical to their function.

3D chromatin compaction has been shown to influence how several IncRNAs are able to direct their protein partners to target loci across the genome. LncRNA-protein complexes may act in concert with DNA elements and enhancer-promoter looping interactions to mediate the assembly of multigene complexes. A remarkable recent observation is the transcription of a novel class of noncoding RNAs arising from enhancer loci throughout the genome. These enhancer-derived ncRNAS, termed enhancer RNA (eRNA) or activating RNA, display stimulus-dependent activation and are correlated with the transcriptional activation of nearby coding genes. eRNAs have been demonstrated to exert their activity through interaction with subunits of the Mediator complex, whose depletion abrogates enhancer-promoter loops leading to diminished transcription of target genes. A second subset of IncRNAs displaying enhancer-like activity exert their effect by acting as a scaffold for chromatin remodeling complexes that catalyze the trimethylation of histone H3 on lysine 4 (Wang et al, 2011: Gomez et al, 2013) in “pre-configured” chromatin environments. This suggests that IncRNAs may utilize pre-formed 3D chromosomal compaction to direct protein-binding partners to prime interacting genes for gene activation. A number of IncRNAs have been identified that may regulate innate immune gene transcription by directing the necessary proteins to target gene promoters. However, it remains unknown whether similar IncRNA-based mechanisms modulate the function of the CXC chemokines and how 3D nuclear architecture influences uniformity and rapidity of transcriptional activation.

Here we identify and mechanistically characterise a new enhancer-like IncRNA, UMLILO (Upstream Master LncRNA of the Inflammatory chemokine LOcus), which is conserved in higher vertebrates, but lacks a homolog in rats and mice. Using multiple genome-wide and single cell data sets we demonstrate that UMLILO is transcribed within the ˜500 Kbp chemokine TAD and arises from within a previously identified enhancer-dense region or super-enhancer (SE). Functional studies reveal that both super-enhancer and UMLILO activity act independently to drive chemokine transcription. Further, by combining a novel single cell genome-editing approach with population-based assays, we show the mechanism by which UMLILO acts in cis, to coordinate chemokine gene regulation by acting as a guide or scaffold to recruit WDR5 to the chemokine TAD, maintaining H3K4me3 across the chemokine gene promoters. Further, we show that the UMLILO-WDR5 complex is brought in close proximity to the chemokine genes by pre-formed chromosomal loops, which form upstream of UMLILO-mediated activity. Finally—and without precedent—we show that by replacing UMLILO with HOTTIP, a IncRNA which shared molecular partners with UMLILO, in a similar genomic location we are able to restore chemokine gene activation. Collectively, these data reveal that pre-formed chromosomal contact in the chemokine TAD provides the platform that facilitates the formation of a IncRNA-WDR5 complex in close proximity to the chemokine gene promoters. This in turn coordinates the hyper-responsive nature of the chemokine genes leading to robust and uniform transcriptional responses across diverse cell types. Remarkably, IncRNAs able to recruit the WR5-MLL1 complex to the appropriate genomic location can functionally substitute for each other. In sum, it creates a new paradigm for how nuclear architecture and IncRNA regulation assists in the rapid transcription of innate immune genes in inflammatory responses.

### SUMMARY OF INVENTION

The invention relates to compositions and methods of modulating the innate immune response by controlling levels of chemokines secreted by immune (macrophages, dendritic cells, neutrophils, basophils, eosinophils) and non-immune cells (fibroblasts, osteoblasts, epithelial, endothelial cells).

In a first aspect of the invention there is provided for an UMLILO IncRNA inhibitor for use in a method of decreasing the production of ELR+ proinflammatory chemokines in a subject, wherein the method comprises administering an effective amount of the UMLILO IncRNA inhibitor to the subject.

Adjustment of chemokine levels is achieved by increasing or decreasing the activity of UMLILO, a novel long non-coding RNA that induces expression of the proinflammatory chemokines (IL-8, CXCL1, CXCL3 and CXCL2). Further, it will be appreciated that replacing the genomic UMLILO IncRNA gene with a IncRNA gene which encodes a different IncRNA (such as HOTTIP) which is capable of binding WDR5 also leads to the modulation of chemokine levels.

In one embodiment of the invention the ELR+ proinflammatory chemokines are IL8, CXCL1, CXCL2 and CXCL3.

In another embodiment of the invention an UMLILO IncRNA inhibitor may be selected from the group consisting of antisense oligonucleotides, miRNAs, siRNAs, piRNAs, snRNAs, CAS9/CRISPR, TALENS, zinc finger nucleases, BUD1 nucleases, antibody drug conjugates (ADCs) or small molecule inhibitors. Preferably, the siRNA is an siRNA selected from the group consisting of SEQ ID NOs:54 to 57.

Preferably, the small molecule inhibitor may be selected from the group consisting of MM-102, MM401 or OICR-9429.

Adjustment of chemokine levels may be achieved by using the aforementioned small molecule inhibitors that block the interaction between WDR5 and MLL1.

Adjustment of chemokine levels may be achieved by using small molecule inhibitors that block the interaction between WDR5 and other members of the WAR complex (Ash2L, DPY-30, RbBP5).

Adjustment of chemokine levels may be achieved by using small molecule inhibitors that block the interaction between WDR5 and UMLILO.

In particular, the invention relates to the use of inhibitors of UMLILO, recombinant nucleic acids encoding UMLILO, or recombinant UMLILO incorporating nucleoside analogs wherein the recombinant UMLILO does not bind to WDR5, to modulate levels of chemokines for treatment of inflammatory conditions, autoimmune diseases, infections by pathogens (e.g., viruses, bacteria, fungi, and protists or other eukaryotic parasites), immunodeficiency, and cancer.

Exemplary UMLILO inhibitors may include antisense oligonucleotides, inhibitory RNA molecules, such as miRNAs, siRNAs, piRNAs, and snRNAs, and ribozymes. In certain embodiments, the UMLILO inhibitor is a recombinant polynucleotide comprising a promoter operably linked to a polynucleotide encoding an inhibitor of UMLILO.

It will be appreciated that in the instances where the UMLILO IncRNA inhibitor is a polynucleotide that such polynucleotide may be provided by a recombinant polynucleotide comprising a promoter operably linked to a polynucleotide encoding the UMLILO IncRNA inhibitor. Preferably, when the UMLILO IncRNA inhibitor is a polynucleotide or is encoded by a polynucleotide then the UMLILO IncRNA inhibitor may be provided by a lentiviral vector-carrying short hairpin RNA (shRNA) selected from the group consisting of SEQ ID NOs:54 to 57.

In a preferred embodiment the subject displays reduced inflammation after treatment with the UMLILO IncRNA inhibitor. Preferably, the subject has an inflammatory condition, an autoimmune disorder or cancer.

In certain embodiments, the subject has an inflammatory condition or an autoimmune disorder, such as, but not limited to multiple sclerosis, rheumatoid arthritis, stomatitis, lupus erythematosus, ischemic heart disease, atherosclerosis, cancer, fibrosis, autoimmune thyroid disease (AITD), inflammatory bowel disease, inflammatory myopathy, giant cell arteritis (GCA), asthma, allergy, Parkinson's disease, or Alzheimer's disease. In another embodiment, the subject has damaged tissue or a wound.

Typically, the inflammatory condition may be select from the group consisting of allergy, acne vulgaris, appendicitis, asthma, atherosclerosis, bursitis, cancer, celiac disease, chronic prostatitis, colitis, cystitis, dermatitis, glomerulonephritis, hay fever, viral infection, bacterial infection, fungal infection, archeal infection, inflammatory bowel disease, pelvic inflammatory disease, periodontitis, phlebitis, reperfusion injury, rhinitis, rheumatoid arthritis, sarcoidosis, sepsis, tendonitis, tonsillitis, transplant rejection, type 1 hypersensitivity and vasculitis.

Alternatively, in instances where the subject has an autoimmune disorder, the disorder may be selected from the group consisting of acute disseminated encephalomyelitis, acute motor axonal neuropathy, Addison's disease, adiposis dolorosa, adult-onset still's disease, alopecia areata, ankylosing spondylitis, anti-glomerular basement membrane nephritis, anti-neutrophil cytoplasmic antibody-associated vasculitis, anti-N-methyl-D-aspartate receptor encephalitis, antiphospholipid syndrome, antisynthetase syndrome, aplastic anemia, autoimmune angioedema, autoimmune enteropathy, autoimmune hemolytic anemia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenic purpura, autoimmune thyroiditis, autoimmune urticarial, autoimmune uveitis, balo concentric sclerosis, Behcet's disease, Bickerstaff's encephalitis, bullous pemphigoid, celiac disease, chronic inflammatory demyelinating polyneuropathy, Churg-Strauss syndrome, cicatricial pemphigoid, Cogan syndrome, cold agglutinin disease, crest syndrome, Crohn's disease, dermatitis herpetiformis, dermatomyositis, diabetes mellitus type 1, discoid lupus erythematosus, drug-induced lupus, endometriosis, enthesitis-related arthritis, eosinophilic fasciitis, epidermolysis bullosa acquisita, erythema nodosum, essential mixed cryoglobulinemia, Evans syndrome, Felty syndrome, fibromyalgia, gestational pemphigoid, giant cell arteritis, Graves' disease, Graves ophthalmopathy, Guillain-Barré syndrome, Hashimoto's encephalopathy, Henoch-Schonlein purpura, hidradenitis suppurativa, idiopathic inflammatory demyelinating diseases, Igg4-related systemic disease, inclusion body myositis, intermediate uveitis, interstitial cystitis, juvenile arthritis, Kawasaki's disease, Lambert-Eaton myasthenic syndrome, leukocytoclastic vasculitis, lichen planus, lichen sclerosus, ligneous conjunctivitis, linear IgA disease, lupus nephritis, lupus vasculitis, Lyme disease, Meniere's disease, microscopic colitis, microscopic polyangiitis, mixed connective tissue disease, Mooren's ulcer, morphea, Mucha-Habermann disease, multiple sclerosis, myasthenia gravis, myocarditis, myositis, neuromyelitis optica, neuromyotonia, opsoclonus myoclonus syndrome, optic neuritis, Ord's thyroiditis, palindromic rheumatism, paraneoplastic cerebellar degeneration, paroxysmal nocturnal hemoglobinuria, Parry Romberg syndrome, Parsonage-Turner syndrome, pediatric autoimmune neuropsychiatric disorder associated with streptococcus, pemphigus vulgaris, pernicious anemia, pityriasis lichenoides et varioliformis acuta, polyarteritis nodosa, polymyalgia rheumatica, polymyositis, postmyocardial infarction syndrome, primary biliary cirrhosis, primary sclerosing cholangitis, progressive inflammatory neuropathy, psoriasis, psoriatic arthritis, pure red cell aplasia, reactive arthritis, relapsing polychondritis, restless leg syndrome, retroperitoneal fibrosis, rheumatic fever, rheumatoid arthritis, rheumatoid vasculitis, sarcoidosis, Schnitzler syndrome, scleritis, Sjogren's syndrome, stiff person syndrome, subacute bacterial endocarditis, Susac's syndrome, Sydenham chorea, sympathetic ophthalmia, systemic lupus erythematosus, systemic scleroderma, thrombocytopenia, Tolosa-Hunt syndrome, transverse myelitis, ulcerative colitis, undifferentiated connective tissue disease, urticarial vasculitis, vasculitis and vitiligo

In a second aspect of the invention there is provided for an UMLILO IncRNA for use in a method of increasing the production of ELR+ proinflammatory chemokines in a subject, wherein the method comprises administering an effective amount of an UMLILO IncRNA to the subject, wherein transcription of the ELR+ proinflammatory chemokines is initiated in a cell when WRD5 is present with the UMLILO IncRNA.

It will be appreciated that in the UMLILO IncRNA inhibitor may be provided to the subject or a cell by a recombinant polynucleotide comprising a promoter operably linked to a polynucleotide encoding the UMLILO IncRNA.

In a preferred embodiment, the subject has an immunodeficiency and the immunodeficiency results in the subject having an increased risk of a pathogenic infection. Preferably, the pathogenic infection is a viral, bacterial, fungal or parasitic infection.

A further aspect of the invention provides for methods of decreasing the production of ELR+ proinflammatory chemokines in a subject, comprising administering an effective amount of an UMLILO IncRNA inhibitor to the subject.

It will be appreciated that the method of the invention results in a subject displaying reduced inflammation after treatment with the UMLILO IncRNA inhibitor.

The method of the invention finds application in instances where the subject has an inflammatory condition, an autoimmune disorder or cancer.

In a fourth aspect of the invention there is provided for a method of increasing the production of ELR+ proinflammatory chemokines in a subject, the method comprising administering an effective amount of an UMLILO IncRNA to the subject, wherein transcription of the ELR+ proinflammatory chemokines is initiated in a cell when WRD5 is present with the UMLILO IncRNA. Typically, the method of increasing the production of proinflammatory cytokines will be used instances where the subject has an immunodeficiency and wherein the immunodeficiency results in the subject having an increased risk of a pathogenic infection.

In a fifth aspect of the invention there is provided for a method of increasing transcription of an UMLILO IncRNA in a cell, comprising administering a compound selected from the group consisting of calcium, nuclear factor of activated T-cells (NF-AT) or activator protein 1 (AP1) to the cell, wherein transcription of UMLILO in the cell is increased after administration of the compound.

The invention also includes a method of decreasing production of ELR+ chemokines by immune and non-immune cells in a subject, the method comprising administering an effective amount of a UMLILO inhibitor or a vector encoding a UMLILO inhibitor to the subject.

In another aspect, the invention includes a method for treating an infectious disease comprising administering to a subject in need thereof a therapeutically effective amount of UMLILO.

In another aspect, the invention includes a method for treating an inflammatory condition or autoimmune disorder comprising administering to a subject in need thereof a therapeutically effective amount of at least one UMLILO inhibitor.

In another aspect, the invention includes a method for inhibiting UMLILO in a subject comprising administering an effective amount of a UMLILO inhibitor to the subject.

### BRIEF DESCRIPTION OF THE FIGURES

Non-limiting embodiments of the invention will now be described by way of example only and with reference to the following figures:

FIG. 1: UMLILO is a new super-enhancer resident IncRNA that is transcribed within the ELR+ CXC chemokine TAD: Hi-C analysis across the CXC chemokine locus reveals that the chemokine TAD spans a region of ˜500 Kbp.

FIG. 2: UMLILO is a new super-enhancer resident IncRNA that is transcribed within the ELR+ CXC chemokine TAD: ChIA-PET data from a library constructed in HUVECs with an antibody that enriches for actively transcribing Pol II. Unstimulated HUVECs displayed relatively few PETs/chromosomal contacts. Upon chemokine gene induction with TNF for 30 mins numerous PETs/contacts were observed, occurring between the super-enhancer region and the chemokine genes, resulting in the formation of a putative ‘inverted rosette’ structure. Contacts detected by ChIA-PET between interacting chemokine genes, were confined exclusively to the region encompassing the chemokine TAD.

FIG. 3: UMLILO is a new super-enhancer resident IncRNA that is transcribed within the ELR+ CXC chemokine TAD: ChIPseq analysis across the chemokine TAD revealed that the SE is atypically large (˜80 Kbp) and highly enriched for H3K4me1 and H3K27Ac epigenetic marks. The UMLILO IncRNA was identified from CAGE data to be expressed in unstimulated endothelial cells. UMLILO does not possess the typical eRNA histone modifications, H3K4me1 and H3K27Ac.

FIG. 4: UMLILO transcription precedes chemokine gene activation: The chemokine TAD structure is formed prior to gene activation by TNF and UMLILO is brought in close proximity to the chemokine genes by pre-formed chromosomal loops.

FIG. 5: UMLILO transcription precedes chemokine gene activation: Intronic smFISH reveals that the chemokines are only induced in HUVECs following TNF induction. Intronic RNA FISH foci of co-expressed chemokine genes always co-localize (n, number of alleles; scale bar, 5 μm; cells were counterstained with DAPI.).

FIG. 6: UMLILO transcription precedes chemokine gene activation: RNA FISH on intron 1 and intron 2 of UMLILO reveal UMLILO is expressed in HUVECs prior to TNF stimulation. The signal from both introns consistently colocalized. Levels of intronic UMLILO were elevated shortly after TNF treatment, and then dropped back down to resting UMLILO levels after longer durations of TNF stimulation (n, number of alleles; scale bar, 5 μm; cells were counterstained with DAPI.).

FIG. 7: UMLILO transcription precedes chemokine gene activation: Simultaneous intronic RNA FISH on UMLILO and the chemokine genes revealed that co-localization between intronic UMLILO and the chemokine genes was rarely observed (n, number of alleles; scale bar, 5 μm; cells were counterstained with DAPI.).

FIG. 8: UMLILO transcription precedes chemokine gene activation: UMLILO is highly conserved in higher vertebrates with neutrophil rich blood, but no homolog of UMLILO or IL8 exists in mice.

FIG. 9: UMLILO transcription is not influenced by perturbing transcriptional regulators that densely occupy the super-enhancer, but is necessary for chemokine transcription: The super-enhancer is densely occupied by chromatin regulators, including the mediator subunit, MED12, and the chromatin reader BRD4.

FIG. 10: UMLILO transcription is not influenced by perturbing transcriptional regulators that densely occupy the super-enhancer, but is necessary for chemokine transcription: Chemokine expression is sensitive to BRD4 depletion. (+) JQ1 but not its enantiomer (−) JQ1 abrogated chemokine transcriptional activation, but did not influence UMLILO transcription (cells were counterstained with DAPI; scale bar, 5 μm).

FIG. 11: UMLILO transcription is not influenced by perturbing transcriptional regulators that densely occupy the super-enhancer, but is necessary for chemokine transcription: Chemokine expression is sensitive to MED12 depletion. Depleting MED12 abrogates the expression of the CXC chemokines, but did not significantly alter the expression of UMLILO (Mean+/− SD; **p<0.05, ***p<0.01; two-tailed unpaired Student's T test).

FIG. 12: UMLILO transcription is not influenced by perturbing transcriptional regulators that densely occupy the super-enhancer, but is necessary for chemokine transcription:UMLILO is necessary for chemokine expression. siRNA knockdown of UMLILO was sufficient to significantly abrogate chemokine expression; but not another TNF-induced gene located in a different TAD (Mean+/− SD; **p<0.05, ***p<0.01; two-tailed unpaired Student's T test).

FIG. 13: UMLILO acts in cis to regulate chemokine expression: The CRISPR-Cas9 system was used to erase the genomic sequence encoding UMLILO and replace it with an eGFP reporter sequence in primary HUVEC cells.

FIG. 14: UMLILO acts in cis to regulate chemokine expression: Successful repair was detected using RNA FISH probes targeting eGFP RNA, driven by the endogenous UMLILO promoter.

FIG. 15: UMLILO acts in cis to regulate chemokine expression: Successful repair was observed in ˜26% of cells, with ˜19% of cells displaying a single foci, and ˜7% of cells displaying dual eGFP foci. Cells displaying a single eGFP focus were still able to express UMLILO from the opposite allele. The deletion of UMLILO abrogated chemokine expression in cis. Co-localization between eGFP foci and IL8 was never observed (Two-tailed Fisher's exact test; *p<0.01; cells were counterstained with DAPI; Scale bar, 5 μm).

FIG. 16: UMLILO acts in cis to regulate chemokine expression: UMLILO exerts its effect in cis. In cells displaying no eGFP foci, expression of IL8 and CXCL2 was comparable to the mock-transfected cells. In cells displaying a single eGFP focus, the expression of IL8 and CXCL2 was reduced significantly. Chemokine expression was never observed in cells displaying dual eGFP foci (Two-tailed Fisher's exact test; *p<0.01; cells were counterstained with DAPI; Scale bar, 5 μm).

FIG. 17: UMLILO interacts with the WAR complex to maintain H3K4me3 epigenetic regulation of chemokine genes: UMLILO interacts with WDR5 (SEQ ID NO. 315), UMLILO was recovered from the nuclear fraction of cell lysates using biotinylated single stranded DNA probes tiling the exonic portion of the IncRNA. Using the MRMHR mass spectrophotometry approach, we identified WDR5, but not Ash2L, RbBP5 or DY30, in the UMLILO pull downs. Immunofluoresence revealed that WDR5 is a low abundant protein located in the nucleus. Immuno-RNA FISH revealed that distinct foci of WDR5 colocalize with UMLILO. Cyto.=cytoplasmic fraction (*p<0.05, **p<0.01, two-tailed unpaired Student's T test; Scale bar, 5 μm). Peptide 1 is represented by SEQ ID NO: 316, peptide 2 by SEQ ID NO: 317, and peptide 3 by SEQ ID NO: 318.

FIG. 18: UMLILO interacts with the WAR complex to maintain H3K4me3 epigenetic regulation of chemokine genes: UMLILO directs WDR5 to the chemokine promoters to facilitate their epigenetic activation. siUMLILO modulates WDR5 binding as well as Pol II-Ser5 and H3K4me3 occupancy on chemokine promoters (*p<0.05, **p<0.01, two-tailed unpaired Student's T test).

FIG. 19: UMLILO interacts with the WAR complex to maintain H3K4me3 epigenetic regulation of chemokine genes: Targeting MLL1-WDR5 interactions abrogated chemokine transcription. MM102 led to a significant reduction in chemokine gene transcription (*p<0.05, **p<0.01, two-tailed unpaired Student's T test).

FIG. 20: UMLILO does not regulate chromosomal contact across the chemokine TAD: Depleting UMLILO using siRNA does not significantly alter chromosomal contact across the chemokine TAD. ChIA-PET data reveals the sites of active Poi II-mediated chromosomal contacts.

FIG. 21: UMLILO does not regulate chromosomal contact across the chemokine TAD: siUMLILO did not significantly alter the chromosomal contacts between UMLILO and IL8. siGFP was used as a control for the detection of baseline chromosomal contacts.

FIG. 22: The activity of UMLILO can be substituted with a different WDR5-interacting enhancer-like IncRNA: HOTTIP FISH spots were observed at approximately 25% of alleles and only single allelic expression of HOTTIP per HeLa cell was observed (n, number of cells; cells were counterstained with DAPI; Scale bar, 5 μm).

FIG. 23: The activity of UMLILO can be substituted with a different WDR5-interacting enhancer-like IncRNA: Graphical representation of the repair strategy. The repair construct consists of the sequence of HOTTIP flanked by homologous arms that flank UMLILO.

FIG. 24: The activity of UMLILO can be substituted with a different WDR5-interacting enhancer-like IncRNA: Replacing endogenous UMLILO with HOTTIP restores chemokine transcription. Overlapping HOTTIP and chemokine foci were observed in ˜1% of HeLas dual transfected with the CRISPR and HOTTIP repair construct for 120 hr and stimulated with TNF for 24 hrs (n, number of cells; cells were counterstained with DAPI; Scale bar, 5 μm).

FIG. 25: The activity of UMLILO can be substituted with a different WDR5-interacting enhancer-like IncRNA: WDR5-interacting IncRNAs exploit pre-formed 3D chromatin folding to coordinate rapid chemokine gene activation. By acting in cis, WDR5-interacting IncRNAs use the local chromatin compaction of the pre-formed chemokine TAD to direct the WDR5 protein across the chemokine promoters. Upon activation by TNF, the chemokine super-enhancer becomes loaded with BRD4 and other transcriptional regulators, to induce the rapid and robust transcriptional induction of the chemokine genes.

FIG. 26: UMLILO is a novel IncRNA of the human ELR+ chemokine locus that binds directly to the WDR5 protein to recruit the MLL H3K4 methylase complex, maintaining H3K4me3 on the chemokine promoters.

FIG. 27: Tacrolimus inhibits UMLILO and IL-8 expression. The LASAGNA (Length-Aware Site Alignment Guided by Nucleotide Association) algorithm for transcription factor binding site (TFBS) alignment was used to analyze the UMLILO promoter for putative TFBS. Transcription Factor Binding Sites screened include NF-AT1 (SEQ ID NO: 319), NF-AT2 (SEQ ID NO: 320), NF-IL6-2 (SEQ ID NO: 321), GATA-3 (SEQ ID NO: 322), SRF (SEQ ID NO: 323) and ATF-2 (SEQ ID NO: 324). A high confidence binding site for the calcium dependent transcription factors, NF-AT1 and NF-AT2 was observed near to the TSS of UMLILO.

FIG. 28: Tacrolimus inhibits UMLILO and IL-8 expression. CHIP assays confirm that the NF-AT transcription factors occupy the UMLILO promoter.

FIG. 29: Tacrolimus inhibits UMLILO and IL-8 expression. Tacroliumus, a macrolide calcium inhibitor, leads to a reduction in UMLILO and IL-8 expression.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

As used herein the term “UMLILO” refers to the Upstream Master LncRNA of the Inflammatory chemokine LOcus, a long non-coding RNA (IncRNA) encoded by the human genome sequence corresponding to the following genomic co-ordinates: chr4:74,576,019-74,580,0244. An exemplary DNA sequence encoding for UMLILO is provided in SEQ ID NO:1. Alternative transcript names for UMLILO include: TCONS_00007551; ENST00000436089; ENST00000436089.1; AC112518.3-001; OTTHUMT00000322 216.2; NONHSAT096866.

The term “immunomodulatory” or “modulating an immune response” as used herein includes immunostimulatory as well as immunosuppressive effects. Immunomodulation, for example, by UMLILO or an inhibitor of UMLILO may cause an increase or decrease in CXC production, respectively, in an individual treated in accordance with the methods of the invention as compared to the absence of treatment. The level of CXC, in turn modulates chemotaxis innate and cellular immune responses by interacting with G protein-linked transmembrane receptors or chemokine receptors, that are selectively found on the surfaces of their target cells e.g. neutrophils, macrophages, T-lymphocytes, mast cells and eosinophils.

The term “microRNA” refers to endogenous or artificial non-coding RNAs that are capable of regulating gene expression. It is believed that miRNAs function via RNA interference. Endogenous (e.g., naturally occurring) miRNAs are typically expressed from RNA polymerase II promoters and are generated from a larger transcript.

The term “siRNA” refers to single-stranded or double-stranded RNA molecules that are capable of inducing RNA interference. siRNA molecules typically have a duplex region that is between 18 and 30 base pairs in length.

The term “piRNA” refers to a class of small RNAs involved in gene silencing. piRNA molecules typically are between 26 and 31 nucleotides in length.

The term “snRNA” refers to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against the IncRNA, UMLILO.

The terms “chemokine,” “pro-inflammatory chemokine,” “CXC chemokine” and “CXC” are interchangeable and refer to cytokines characterised according to behavioural and structural characteristics to induce neutrophil chemotaxis to the site of injury or infection, in particular CXC chemokines having two N-terminal cysteines separated by one amino acid, even more particularly having a specific amino acid sequence of glutamic acid-leucine-arginine (ELR) immediately before the first cysteine of the CXC motif (ELR-positive or ELR+ CXC chemokines). ELR-positive (ELR+) CXC chemokines specifically induce the migration of neutrophils, and interact with chemokine receptors CXCR1 and CXCR2. An example of an ELR-positive CXC chemokine is interleukin-8 (IL-8), which induces neutrophils to leave the bloodstream and enter into the surrounding tissue.

The term “exon” when used in relation to a IncRNA refers to a defined section of nucleic acid that is represented in the mature form of a IncRNA molecule after portions of a pre-processed (or precursor) IncRNA have been removed by splicing.

The term “intron” when used in relation to a IncRNA refers to a nucleic acid region in the precursor IncRNA that is subsequently removed by splicing during formation of the mature IncRNA.

The terms “polynucleotide,” “oligonucleotide,” “oligo” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. There is no intended distinction in length between these terms and they are used interchangeably.

The term “pharmaceutically acceptable excipient or carrier” refers to an excipient that may optionally be included in the compositions of the invention and that causes no significant adverse toxicological effects to the patient.

The term “pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

The term “pathogen” or “parasite” or “microbe” refers to any virus or organism that spends at least part of its life cycle or reproduces within a host. Intracellular pathogens include, but are not limited to, viruses (e.g., influenza virus, respiratory syncytial virus, hepatitis virus B, hepatitis virus C, herpes virus, papilloma virus, and human immunodeficiency virus), bacteria (e.g., Listeria, Mycobacteria (e.g., Mycobacterium tuberculosis, Mycobacterium leprae), Salmonella (e.g., S. typhi), enteropathogenic Escherichia coli (EPEC), enterohaemorrhagic Escherichia coli (EHEC), Yersinia, Shigella, Chlamydia, Chlamydophila, Staphylococcus, Legionella), protozoa (e.g., Plasmodium (e.g., P. vivax, P. falciparum, P. ovale, and P. malariae), Taxoplasma, Leishmania), and fungi (e.g., Aspergillus, Blastomyces, Candida). Eukaryotic intercellular parasites include trematodes (e.g., Schistosoma, Clonorchis), hookworms (e.g., Ancylostoma duodenale and Necator americanus), and tape worms (e.g., Taenia solium, T. saginata, Diphyllobothrium spp., Hymenolepis spp., Echinococcus spp.).

The term “cancer” refers to human cancers and carcinomas, sarcomas, adenocarcinomas, lymphomas, leukemias, etc., including but not limited to solid tumors and lymphoid cancers, kidney, breast, lung, kidney, bladder, colon, ovarian, prostate, pancreas, stomach, brain, head and neck, skin, uterine, testicular, esophagus, and liver cancer, lymphoma, including non-Hodgkin's and Hodgkin's lymphoma, leukemia, and multiple myeloma.

The terms “overexpress,” “overexpression” or “overexpressed” interchangeably refer to a gene that is transcribed or translated at a detectably greater level, in comparison to a normal cell. Overexpression therefore refers to both overexpression of protein and/or RNA (due to increased transcription, post transcriptional processing, translation, post translational processing, altered stability, and/or altered protein degradation), as well as local overexpression due to altered protein traffic patterns (increased nuclear localization), and augmented functional activity, e.g., as in an increased enzyme hydrolysis of substrate.

The term “inhibitor” refers to an agent that, by way of non-limiting example, inhibits expression of a IncRNA of the invention or bind to, partially or totally block stimulation or enzymatic activity, decrease, prevent or delay activation, inactivate, desensitize, or down regulate the activity of a IncRNA of the invention, e.g., antagonists.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “up-regulation” refers to the process by which a cell increases the quantity of a cellular component, such as RNA or protein, in response to an internal or external variable or stimulus.

An “effective amount” of UMLILO is an amount sufficient to effect beneficial or desired results, such as an amount that increases production of CXCs. An effective amount can be administered in one or more administrations, applications, or dosages.

An “effective amount” of an UMLILO inhibitor (e.g., microRNA, siRNA, piRNA, snRNA, antisense nucleic acid, ribozyme, or small molecule inhibitor) is an amount sufficient to effect beneficial or desired results, such as an amount that inhibits the activity of UMLILO, for example by interfering with transcription of UMLILO, binding of UMLILO to WDR5, or activation of CXC gene expression. An effective amount can be administered in one or more administrations, applications, or dosages.

Statistics

p values from two-tailed unpaired Student's t test and Fisher Exact tests were calculated using R. *p<0.05, **p<0.01, and ***p<0.001.

Bioinformatic analysis of both Hi-C and ChIA-PET data in the CXC TAD identified a ‘super-enhancer’ region, spanning ˜80 kb and forming extensive chromosomal contacts with the proinflammatory genes. CAGE analysis across various primary cell types and cell lines identified UMLILO (upstream master linc of the chemokine locus), a novel IncRNA transcribed from within the super enhancer region in resting immune and non-immune cells. UMLILO is highly conserved in mammals with neutrophil-rich blood, whereas no homolog of either UMLILO or IL-8 exists in rodents and other animals with neutrophil-poor blood. “Super enhancer” drugs targeting BRD4 abrogate chemokine expression, but UMLILO expression remained unaffected. Using genome-editing technologies and siRNA knockdown approaches we show that UMLILO is necessary for chemokine expression. Further, simultaneous intronic FISH revealed that UMLILO transcription precedes the chemokine gene expression. This suggests that UMLILO may be required to establish a nuclear compartment that facilitates the rapid activation of chemokine gene expression. Accordingly, RNA immunopreciptation experiments revealed that UMLILO might coordinate chemokine gene regulation by acting as a scaffold to recruit chromatin remodelers and the WAR complex to the chemokine TAD. Preventing the WDR5/MLL1 interaction using the small molecule inhibitor, MM-102 abrogated chemokine transcription. Therefore, we present a novel approach to alter the transcriptional status of chemokine genes.

The following examples are offered by way of illustration and not by way of limitation.

### Example 1

Cell Culture

Early passage HUVECs from pooled donors (Lonza) were grown to ˜80% confluence in Endothelial Basal Medium-2 (EGM-2) with supplements (Lonza), serum-starved (18 hr) in EGM-2+0.5% FBS, and treated with TNF (10 ng/ml; Sigma) for up to 24 hr. Prior to transfection cells were grown in antibiotic free EGM-2.

Hi-C Analysis

Hi-C sequencing data were preprocessed (iterative alignment and outlier removal) using the pipeline described by Imakaev and colleagues (Imakaev et al. 2012). The heatmap in the chemokine locus show the Hi-C interactions as paired-read counts between pairs of sliding windows of 50 Kbp in length.

ChIA-PET Analysis

The two Pol II (Ser2/Ser5) ChIA-PET libraries for HUVEC were obtained from NCBI (accession numbers: GSE41553) (Papantonis et al. 2012). One library is obtained 0 min after TNF treatment while another one is obtained 30 min after TNF treatment. Each library yielded about 35 millions paired-end reads. Both libraries were processed by ChIA-PET tool (Li et al. 2010). Briefly, the head and tail ChIA-PET tags are first extracted from the sequences. The ChIA-PET tags were then mapped to the human reference genome (hg19) by Batmis (Tennakoon et al. 2012) and 10.8×10⁶ and 8.8×10⁶ are successfully aligned to hg19 for 0 min and 30 min samples, respectively. From the uniquely mapped paired-end tags (PETs), we extract all intra-chromosomal PETs. Precisely, intra-chromosomal PETs were defined as the head and tail of the PETs mapped onto the same chromosome with a genomic distance at least 5 Kbp. The intra-chromosomal PETs are then clustered to form ChIA-PET interaction clusters.

Identification of Enhancers in the Chemokine TAD

We used the web tool PrESSto (Promoter Enhancer Slider Selector Tool, htp://pressto.binf.ku.dk/) to query the atlas of active, in vivo bi-directionally transcribed human enhancers identified analysing the FANTOMS panel of tissue and primary cell samples (Andersson et al. 2014). Across the chemokine locus (chr4: 74,500,000-75,000,000) we searched for enhancers significantly expressed in the “blood vessel endothelial cell” and the “macrophage” facets (% expression≥1, P value<0.001, Benjamini-Hochberg adjusted FDR<0.05). Every identified enhancer was associated to its overall % of expression and number of tags per million (tpm) in the facets, as well as its relative tpm in every single sample belonging to the analysed facets.

Identification of Long Noncoding RNAs in the Chemokine TAD

We used the ENSEMBL mart human database (Release GRCh37.p13) to identify all the IncRNAs mapping to the chemokine locus (chr4:74,500,000-75,000,000). We then queried the FANTOMS human expression atlas to evaluate the promoter activity associated to the Transcription Start Site (TSS) of every identified IncRNA. The ZENBU omics interactive visualization system (human hg19, February 2015 updated release) was used to derive the expression levels of each IncRNA in 1829 human samples (primary cells, cancer cell-lines and post-mortem tissues) profiled either in steady state or time-course experiments.

CRISPR Synthesis

Software developed by the Zhang laboratory was used to identify CRISPR candidate guide sequences (Cong et al. 2013). CRISPRs were generated using the protocol described by Cong et al., briefly, 1 μg of pX330 was digested with Bbsl for 30 min at 37° C., gel purified using QIAquick Gel Extraction Kit and eluted in elution buffer. Oligos were then phosphorylated using T4 PNK (NEB) and annealed using the following parameters: 37° C. for 30 min; 95° C. for 5 min and then ramp down to 25° C. at 5° C./min. Annealed oligos were then ligated into the BbSI-digested pX330 vector using Quickligase (NEB). The ligation reaction was then treated with PlasmidSafe exonuclease to prevent unwanted recombination products at 37° C. for 30 min. Colony PCR was used on E. coli transformants to identify successful CRISPR clones. HUVECs were then microporated with the respective CRISPR using the Neon® Transfection System (Life Technologies) according to manufacturer's instructions. HeLas were transfected with Lipofectamine 2000 according to manufacturer's instructions. Nuclease activity was assessed by the surveyor assay.

RNA Interference

HUVECs and HeLas were transfected as per manufacturer's instructions with siRNAs targeting UMLILO (Dharmacon) using the Neon® Transfection System (Life Technologies) and Lipofectamine 2000 (Invitrogen) respectively. Total RNA was harvested 48-72 hr later using TRIzol (Invitrogen) according to manufacturer's instructions. The following siRNA's were used:

UMLILO siRNA 1:
(SEQ ID NO: 54)
5′ AUC UUA AAU UAG AGG CGA AUU 3′
UMLILO siRNA 2:
(SEQ ID NO: 55)
5′ CAU ACA AAU UCU CGC AGC AUU 3′
UMLILO siRNA 3:
(SEQ ID NO: 56)
5′ AAG AGU UGG UAC ACG GUG AUU 3′
UMLILO siRNA 4:
(SEQ ID NO: 57)
5′ GCA UAU UAA CCC UAC AAG UUU 3′

RT-qPCR

For all RT-qPCR analyses, whole RNA was extracted using TRIzol (Invitrogen) according to manufacturer's instructions. cDNA was generated using the SuperScript III cDNA kit (Invitrogen). All qPCR primers (Table 1) were verified to produce specific products and to perform efficiently.

qPCR reactions were performed with technical triplicates on a CFX Real Time PCR detection system (Biorad) with SsoFast qPCR Supermix (Biorad). Relative quantification was performed using the delta delta Ct method with HPRT as a reference gene.

TABLE 1
QPCR primers

| | Direc- | | SEQ ID |
|---|---|---|---|
| Target | tion | Primer sequence | NO. |
| UMLILO | FW | 5′ GGAAGGAGGGGAACATGGAA 3′ | SEQ ID |
| intron | | | NO: 2 |
| 1 | RW | 5′ CTACCTGACTCCCTCCCTCT 3′ | SEQ ID |
| | | | NO: 3 |
| UMLILO | FW | 5′ GGTACACGGTGAACATTTGCT 3′ | SEQ ID |
| exon 3 | | | NO: 4 |
| | RW | 5′ CAGCATCTCTCTGTCCACTGA 3′ | SEQ ID |
| | | | NO: 5 |
| IL8 | FW | 5′ AGGGCCAAGAGAATATCCGA 3′ | SEQ ID |
| gene | | | NO: 6 |
| | RW | 5′ GGACTTGTGGATCCTGGCTA 3′ | SEQ ID |
| | | | NO: 7 |
| CXCL1 | FW | 5′ AGCTTGCCTCAATCCTGCAT 3′ | SEQ ID |
| gene | | | NO: 8 |
| | RW | 5′ CCTCCTCCCTTCTGGTCAGT 3′ | SEQ ID |
| | | | NO: 9 |
| CXCL2 | FW | 5′ CACAGTGTGTGGTCAACATTTCTC 3′ | SEQ ID |
| gene | | | NO: 10 |
| | RW | 5′ ACACAGAGGGAAACACTGCATAA 3′ | SEQ ID |
| | | | NO: 11 |

RNA FISH Probes

RNA FISH was performed according to the protocol by Raj et al. 2008 using 48 20-mer probes (Biosearch) targeting the following genes: IL-8 intron 1 (SEQ ID NOs:58-101), CXCL1 intron 1 (SEQ ID NOs:187-227), CXCL2 intron 1 (SEQ ID NOs:149-186), UMLILO intron 1 and intron 2 (SEQ ID NOs:260-294) and 28 20-mer probes targeting eGFP (SEQ ID NOs:228-259). Each 20-mer bares a 3′-amino-modifier C6-dT. The amino group was subsequently conjugated to the following NHS-ester dyes: ATTO-488, ATTO-565, ATTO-647N (ATTO-TEC) or Alexa Fluor 647 (Invitrogen). Briefly, oligonucleotide probes were ethanol precipitated and resuspended in 0.1 M sodium tetraborate (Sigma). Approximately 0.3 mg of the NHS-ester dye (ATTO-TEC) was dissolved in dimethyl sulphoxide (Sigma). The dye solution was added to the probe solution and incubated overnight in the dark at 37° C. Following conjugation reaction, the probes were ethanol precipitated overnight, and resuspended in 0.1 M Triethyl ammonium (TEA, Sigma). Conjugated probes were separated and purified to enrich for dye-conjugated probes by reverse phase HPLC on a C18 column.

Immuno-RNA FISH

For each experiment, early passage HUVECs on coverslips were grown to ˜80% confluence, treated with TNF, fixed in 3.7% formaldehyde for 10 mins at room temperature, then washed three times in PBS. Cells were permeabilized in ice-cold 90% methanol for ten minutes, then washed twice with PBS and incubated in blocking buffer (1% BSA/PBS) for 30 minutes at room temperature on an orbital shaker. Cells were then incubated in primary antibody solution (diluted in 1% BSA/PBS) for 1 hr. Double strand breaks were detected with rabbit polyclonal anti-phospho-histone H2A.X (Ser139) (Sigma). Goat polyclonal anti-WDR5 (SantaCruz) was used to detect WDR5 protein. Coverslips were then washed 5 times with wash buffer (0.05% Tween-20/PBS), following incubation with secondary antibodies conjugated to either Atto-488 or Atto-565 for 1 hr. Coverslips were then washed 5 times with wash buffer (0.05% Tween-20/PBS), and post-fixed with 3.7% formaldehyde/PBS for 10 minutes at room temperature, followed by further permeabilization in 70% ethanol overnight. For RNA FISH detection, coverslips were washed twice in PBS and incubated in wash buffer (10% formamide, 2×SCC-1×SCC is 0.15 M NaCl plus 0.015 M sodium citrate) for 5 min. Cells were then hybridized overnight in a humidified chamber at 37° C. in 50 μl of Hyb buffer (10% dextran sulfate, 1 μg/μl E. Coli tRNA, 2 mM Vanadyl ribonucleoside complex, 0.02% RNAse-free BSA, 10% formamide) combined with 50 ng of single molecule FISH probes. Coverslips were then washed 3× (30 min each on the orbital shaker) in wash buffer (10% formamide, 2×SCC). Cells were then incubated in equilibration buffer (0.4% glucose, 2×SCC) for 5 min and counter stained with 1 μg/ml DAPI (4′,6-diamidino-2-phenylindole; Life Technologies). Coverslips were mounted in glox buffer (3.7 μg/μl glucose oxidase, 1 U catalase) and imaged.

Image Acquisition and Processing

Cells were imaged on a custom built Nikon Ti Eclipse widefield TIRF microscope using a 100×N.A. 1.49 Nikon Apochromat TIRF oil immersion objective. Imaging was done using mercury lamp illumination through the appropriate filter sets at low camera gain in each of the fluorescent channels using an Andor iXion897 EMCCD camera cooled to −80° C. The microscope was controlled using μmanager open source microscope management software (NIH and UCSF, USA). A 20 ms exposure time was used for DAPI. Exposure times ranged from 200 to 500 ms for other dyes. Each field of view was captured as a series of images acquired on multiple focal planes through the samples, across a range of 2-10 μm in the axial plane. A 0.2 μm piezo step-size was used for these z-stacks. Chromatic aberration was verified before image capture by alignment of Focal Check Fluorescent Microspheres (Molecular Probes). Signal intensities were measured using Fiji (Schindelin et al., 2012). The contrast of pictures shown was adjusted to fit a 16 bit grey scale. To facilitate the comparison between different fields of view on the same coverslip, IDV values were normalized relative to the intensity of fluorescent beads.

Repair Construct

To generate double stranded PCR product harboring the 20 and 18 bp homologous arms, the following primers were used:

(100 nmole and HPLC purification)

FW: 5′
(SEQ ID NO: 12)
TTGAACCGGGTTTTCCAGTCACATATGGTGAGCAAGGGCGA 3′
RW: 5′
(SEQ ID NO: 13)
ACTATGAAGACTCTTGGGTCACTTGTACAGCTCGTCCA 3′

The PCR product was purified by QIAquick PCR Purification Kit (Qiagen) prior to transfection.

Chromatin Immunoprecipitation

For each ChIP experiment, 1×10⁷ cells were grown to ˜80% confluence, fixed in 3.7% formaldehyde for 10 mins at room temperature, then washed three times in PBS. Then, glycine was added to a final concentration of 125 mM to the media and incubated with shaking for 5 min at RT. Cells were rinsed 2× with 10 ml cold PBS, scraped into 5 ml cold PBS and centrifuged for 5 min at 1,000 g. The resultant pellet was resuspended in FA Lysis Buffer (50 mM HEPES-KOH pH 7.5; 140 mM NaCl; 1 mM EDTA pH 8; 1% Triton X-100; 0.1% Sodium Deoxycholate; 0.1% SDS; Protease Inhibitors (add fresh each time). The nuclear pellet was then sonicated with the Covaris S220 Sonicator to an average fragment size of 500 to 1000 bp and centrifuged for 30 seconds at 4° C., 8,000 g. The supernatant was transferred to a new tube. 50 μl of each sonicated sample was removed as the INPUT to obtain the DNA concentration. 25 μg of protein was used per IP. Protein concentration was calculated using the Bradford assay. Each sample was diluted 1:10 with RIPA buffer (50 mM Tris-HCl pH 8.0; 2 mM EDTA pH 8.0; 1% NP-40; 0.5% Sodium Deoxycholate; 0.1% SDS; Protease Inhibitors (add fresh each time). 2 μg of antibody was added per 25 μg of protein. The following ChIP grade antibodies were used: anti-H3K4me3 (Abcam), MED12 (Bethyl laboratories), anti-RNA Pol II Ser5 (Abcam), anti-WDR5 (H-35 SantaCruz). 20 μl of magnetic protein A/G beads (Resyn Biosciences) (pre-absorbed with sonicated single stranded salmon sperm DNA at 1.5 μg/20 μl beads) to all samples and incubated at 4° C. overnight with rotation. Beads were washed 3× with 1 ml wash buffer (0.1% SDS; 1% Triton X-100; 2 mM EDTA pH 8; 150 mM NaCl; 20 mM Tris-HCl pH 8) and then 1× with final wash buffer (0.1% SDS; 1% Triton X-100; 2 mM EDTA pH 8; 500 mM NaCl; 20 mM Tris-HCl pH 8). DNA was eluted with 120 μl of Elution Buffer (1% SDS; 100 mM NaHCO₃) to the protein A/G beads and incubated at 37° C. for 15 min with rotation. DNA from ChIP and INPUT samples was purified using phenol:chloroform extraction and ethanol precipitated in presence of 10 μl glycogen (5 mg/ml) and taken up in 100 μl H₂O.

TABLE 2
ChIP primers

| | Direc- | | SEQ ID |
|---|---|---|---|
| Target | tion | Primer sequence | NO. |
| IL8 | FW | 5′ TGGGCCATCAGTTGCAAATC 3′ | SEQ ID |
| | | | NO: 14 |
| | RW | 5′ AGTGAGATGGTTCCTTCCGG 3′ | SEQ ID |
| | | | NO: 15 |
| CXCL1 | FW | 5′ ACGTGGGTCTAAGGGATCTG 3′ | SEQ ID |
| | | | NO: 16 |
| | RW | 5′ GGGTCTGACTGTCTTGCGTA 3′ | SEQ ID |
| | | | NO: 17 |
| CXCL2 | FW | 5′ CTGTGGTGGTTCTCAGGGAT 3′ | SEQ ID |
| | | | NO: 18 |
| | RW | 5′ TGGACTCTGAGACTCTGGGA 3′ | SEQ ID |
| | | | NO: 19 |
| CXCL3 | FW | 5′ TCTGGAATCCGAGACGATGG 3′ | SEQ ID |
| | | | NO: 20 |
| | RW | 5′ GACAGGAAAGGCACGACTTC 3′ | SEQ ID |
| | | | NO: 21 |
| SAMD4A | FW | 5′ GAGCTTTGGGTGGAGAGAGT 3′ | SEQ ID |
| | | | NO: 22 |
| | RW | 5′ TTACTCCTCCTCCTCCTCCC 3′ | SEQ ID |
| | | | NO: 23 |
| UMLILO | FW | 5′ TGTCCAAATCCACATTGACAGT 3′ | SEQ ID |
| | | | NO: 24 |
| | RW | 5′ GGAGTGTTGCTGCGAGAATT 3′ | SEQ ID |
| | | | NO: 25 |

RNA Immunoprecipitation with Targeted Mass Spectrophotometry

For each experiment, 1×10⁷ cells were grown to ˜80% confluence, then washed three times in PBS. Briefly, cells were resuspended in Buffer A (10 mM HEPES pH 7.5, 1.5 mM MgCl₂, 10 mM KCl, 0.5 mM DTT, 1.0 mM PMSF), lysed in 0.25% NP40, and fractionated by low speed centrifugation. The nuclear fraction was resuspended and lysed in Buffer C (20 mM HEPES pH 7.5, 10% glycerol, 0.42 M KCl, 4 mM MgCl₂, 0.5 mM DTT, 1.0 mM PMSF). Either the nuclear or cytoplasmic fraction was incubated with biotinylated oligonucleotide probes (SEQ ID NOs:295-314) tiling exonic UMLILO in hyb buffer (100 mM Tris-HCl pH 7.0; 500 mM NaCl; 10 mM EDTA pH 8.0; 15% formamide; 1 mM DTT; 1% SDS; 0.1 U/μl Superase) at 37° C. for 4 hr. 20 μl of magnetic protein A/G beads (Resyn Biosciences) (pre-absorbed with sonicated single stranded salmon sperm DNA at 1.5 μg/20 μl beads) was added to all samples and incubated at 37° C. for 1 hr with rotation. Beads were washed 3× with wash buffer (2×SCC; 0.5% SD; 1 mM DTT). Proteins were eluted with SDS loading buffer and subject to SDS-PAGE analysis. Gels were stained using Colloidal Coomasie and protein bands of interest were in-gel trypsin digested as per the protocol described in Shevchenko et al., 2007. In short, gel bands were destained using 50 mM NH₄HCO₃/50% MeOH followed by in-gel protein reduction (50 mM DTT in 25 mM NH₄HCO₃) and alkylation (55 mM iodoacetamide in 25 mM NH₄HCO₃). Proteins were digested overnight at 37° C. using 5-50 μl, 10 ng/μl tryspin depending on the gel piece size. Digests were resuspended in 40 μl, 2% acetonitrile/0.2% formic acid and analysed using a Dionex Ultimate 3000 RSLC system coupled to an AB Sciex 6600 TripleTOF mass spectrometer. Peptides were first de-salted on an Acclaim PepMap C18 trap column (75 μm×2 cm) for 8 min at 5 μl/min using 2% acetonitrile/0.2% formic acid, then separated on Acclain PepMap C18 RSLC column (75 μm×15 cm, 2 μm particle size). Peptide elution was achieved using a flow-rate of 500 nl/min with a gradient of 4-60% B in 30 min (A: 0.1% formic acid; B: 80% acetonitrile/0.1% formic acid). Nano-spray was achieved using a NanoSpray III source assembled with a New Objective, PicoTip emitter. An electrospray voltage of 2.3 kV was applied to the emitter. Precursor spectra were acquired in the range 400-1500 m/z (0.25 s accumulation time). Product ion spectra were acquired for 18 precursors reporting on 5 targeted proteins. Product scans were recorded in the mass range 100-1500 m/z, high sensitivity mode, with an accumulation time of 0.1 s for a total cycle time of 2.1 s. Data processing was performed in Skyline (Shevchenko et al. 2007). MS1 filtering was performed at 30,000 whilst MS2 at 15,000 resolution with a match tolerance of 0.025 m/z.

3C Assay

3C experiments were performed as described previously by Hagege et al. with some minor modifications (Hagege et al. 2007). Briefly, a single-cell suspension of 8×10⁶ HeLa cells was crosslinked with 1% formaldehyde in 10% (v/v) FBS/PBS for 10 min at room temperature and quenched by adding 0.125 M glycine and incubating for 5 min on ice. Cells were then pelleted by centrifugation at 225 g for 8 min at 4° C. and lysed on ice in 3C lysis buffer (10 mM Tris-HCl, pH 7.5; 10 mM NaCl; 0.2% NP-40; 1× complete protease inhibitor (Roche) for 30 min and subsequently dounce homogenized. Nuclei were collected by centrifugation at 400 g for 5 min at 4° C. The pellets were resuspended in 1.2× Buffer R (Thermo Scientific) with 0.3% SDS and incubated at 37° C. for 1 hr while being shaken at 250 rpm. The SDS was sequestered by adding 2% Triton X-100 and incubating this for 1 hr at 37° C. with shaking. 550 U of HindIII (10 U/ul) (Thermo Scientific) was then added and incubated overnight at 37° C. with shaking. The digestion was terminated with 1.3% SDS at 65° C. for 20 min. 6.125 ml of 1.15× T4 Ligation Buffer (Thermo Scientific) with 1% Triton X-100 was added and incubated at 37° C. for 1 hr with mild shaking (150 rpm). Samples were allowed to cool to room temperature before 100 U T4 DNA ligase (Thermo Scientific) was added and incubated at 16° C. for 4 hr, with gentle shaking (90 rpm), followed by an additional 30 min at room temperature. Crosslinks were reversed with 300 μg of proteinase K and overnight incubation at 65° C. The following day, 300 μg of RNase A was added to the sample and incubated at 37° C. for 30 min. Finally, genomic DNA was purified by phenol chloroform extraction followed by isopropanol precipitation. Ligation events were detected using unidirectional, fragment specific primers (Table 3). qPCR reactions were performed with technical triplicates on a CFX Real Time PCR detection system (Biorad) with SsoFast qPCR Supermix (Biorad). A putative genomic interaction was used to correct for variations between 3C library preparations. Interaction frequencies were calculated relative to the random interaction frequencies as determined with a BAC template (RPCI-11 447E20) spanning the genomic region of interest.

TABLE 3
3C primers

| | Fragment | | |
|---|---|---|---|
| Primer | location | Sequence | SEQ ID NO |
| Anchor | chr4:74604292- | 5′ TCCTCTGACATAATGAAAAGATGAGGGTGC 3′ | SEQ ID NO: 26 |
| | 74606315 | | |
| 1 | chr4:74499669- | 5′ AAAATAGAAACCCTGAATGTACCGGTAACA 3′ | SEQ ID NO: 27 |
| | 74501601 | | |
| 2 | chr4:74511769- | 5′ TCAAATCCGTGATCAGCATTACCAAGCCAT 3′ | SEQ ID NO: 28 |
| | 74524359 | | |
| 3 | chr4:74527725- | 5′ ATTGGAAGGCTAAATATACTTACATGGC 3′ | SEQ ID NO: 29 |
| | 74529421 | | |
| 4 | chr4:74530103- | 5′ TTTTAAAAGCAGCACTAGTGTATCCGG 3′ | SEQ ID NO: 30 |
| | 74534800 | | |
| 5 | chr4:74546061- | 5′ AAAGAGCTCAATGTGTGTTACTAAAGAATG 3′ | SEQ ID NO: 31 |
| | 74548099 | | |
| 6 | chr4:74549704- | 5′ AAAGGTAAGCAATAATTGGCCCATATCTC 3′ | SEQ ID NO: 32 |
| | 74551014 | | |
| 7 | chr4:74553813- | 5′ AAAAGTGATACATGTTCATTGCATTTAAAC 3′ | SEQ ID NO: 33 |
| | 74555419 | | |
| 8 | chr4:74578660- | 5′ CGTAAGGCTGGTCAAGGTATGCTGAG 3′ | SEQ ID NO: 34 |
| | 74585594 | | |
| 9 | chr4:74585594- | 5′ TTCTTCATTATGCTAGTGTTGTGTATTGTG 3′ | SEQ ID NO: 35 |
| | 74587345 | | |
| 10 | chr4:74587345- | 5′ TGAAATTAGAGAAAAACATGTACTTAGGGA 3′ | SEQ ID NO: 36 |
| | 74591147 | | |
| 11 | chr4:74591147- | 5′ CACCCCGCCCATTTGATGAACTGTTT 3′ | SEQ ID NO: 37 |
| | 74596339 | | |
| 12 | chr4:74596459- | 5′ ACTTCAAACTCCAAACTCCACCGATTTG 3′ | SEQ ID NO: 38 |
| | 74601876 | | |
| 13 | chr4:74601963- | 5′ CCAACATCACTGAAGCAAAGAAACTTGGAG 3′ | SEQ ID NO: 39 |
| | 74604292 | | |
| 14 | chr4:74607958- | 5′ TCTGCTTGTACGTAGGTATGTAGATT 3′ | SEQ ID NO: 40 |
| | 74612686 | | |
| 15 | chr4:74722664- | 5′ GCTAGCAACCAACTCTTTAAGAATACAGCC 3′ | SEQ ID NO: 41 |
| | 74725426 | | |
| 16 | chr4:74736063- | 5′ AGAAACACAACGATACAATGTGAAA 3′ | SEQ ID NO: 42 |
| | 74750395 | | |
| 17 | chr4:74751595- | 5′ AGTGTGACTCCAAATAACCTAGTTTGCTA 3′ | SEQ ID NO: 43 |
| | 74756828 | | |
| 18 | chr4:74756828- | 5′ ACACAGTCATAATCACAACCCCAGTC 3′ | SEQ ID NO: 44 |
| | 74759327 | | |
| 19 | chr4:74899318- | 5′ TCAGACTCATGGGCTCAGTTGATTC 3′ | SEQ ID NO: 45 |
| | 74900128 | | |
| 20 | chr4:74901502- | 5′ GGCTGACACATTATGGTCTCCCACTAAATA 3′ | SEQ ID NO: 46 |
| | 74902795 | | |
| 21 | chr4:74903866- | 5′ GAAACATGTCAAGAGGCCGTGGACATTT 3′ | SEQ ID NO: 47 |
| | 74908292 | | |
| 22 | 74917831- | 5′ AGTGTGAAACAAAACGAGAAGGGAAG 3′ | SEQ ID NO: 48 |
| | 74918715 | | |
| 23 | 74964374- | 5′ AAATTATTTGCTTTAGGAAGGGAAGTAGAA 3′ | SEQ ID NO: 49 |
| | 74967509 | | |
| 24 | 74972881- | 5′ CATAGGAGAGACTGCGACAGAAATTCCATT 3′ | SEQ ID NO: 50 |
| | 74981053 | | |
| 25 | 74981053- | 5′ TTACAACTCCTACAACCGTGCTTGGTACAT 3′ | SEQ ID NO: 51 |
| | 74983881 | | |
| GAPDH | chr12:6639882- | 5′ TGCCAATCTCCTTGTTTTCTAATG 3′ | SEQ ID NO: 52 |
| control | 6641136 | | |
| F1 | | | |
| GAPDH | chr12:6641136- | 5′ TATTCCCCCAGGTTTACATGTTC 3′ | SEQ ID NO: 53 |
| control | 6645917 | | |
| F2 | | | |

### Example 2

The ELR+ CXC Chemokines Engage in Pre-Formed Chromosomal Contact

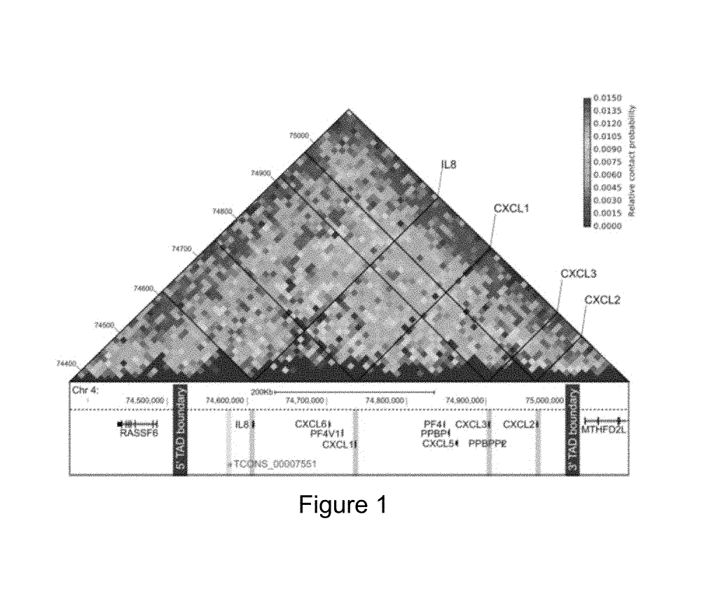

Recent Hi-C and 5C studies show that several classes of innate immune genes are organized into TADs (Jin et al. 2013). Tumor necrosis factor (TNF) has been shown to strongly induce the expression of the ELR+ CXC chemokines (I L8, CXCL1, CXCL2 and CXCL3; hereafter referred to as chemokines) in fibroblasts and endothelial cells (Paulsen et al. 2013; Jin et al. 2013). Therefore, we utilized previously published Hi-C data to examine the higher order chromatin organization of the chemokine TAD in diploid fibroblasts (IMR90), primary endothelial cells (HUVECs) and other cell types. Analysis of Hi-C data across the proinflammatory chemokine locus revealed that the chemokine TAD spans a region of ˜500 Kbp, and is well defined in unstimulated IMR90s, HUVECs and other immune and non-immune cells (FIG. 1). Further, the chemokine TAD in humans may be further divided into two smaller subdomains with the super-enhancer region, IL8 and CXCL1 being located within the same subdomain (˜230 Kbp), whilst CXCL3 and CXCL2 are located within the second sub-domain (˜290 Kbp).

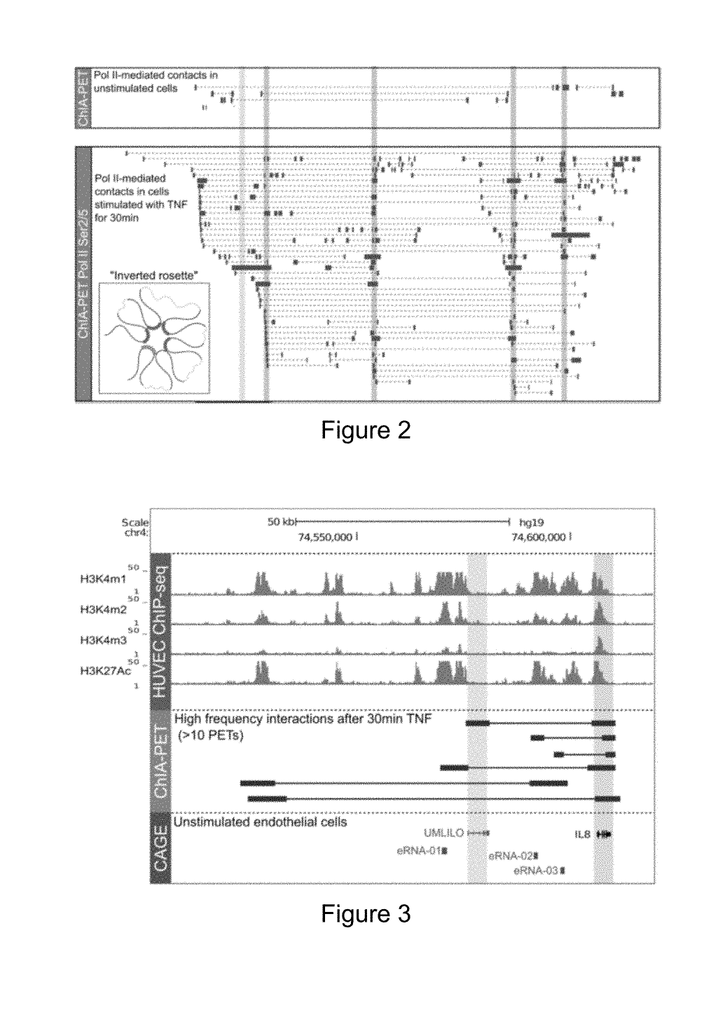

Although Hi-C reveals TAD structure, it does not reveal the status of interacting chromatin at actively transcribing genes. The ChIA-PET technique incorporates a ChIP step, and therefore, enables the detection of chromosomal contacts that are mediated by specific proteins. We analysed ChIA-PET data from a library constructed with an antibody that enriches for actively transcribing RNA Pol II (dual phosphorylated at Ser2/Ser5) (Papantonis et al. 2012). To minimise amplification effects, two or more reads that were either identical, or overlapped by +/−2 bp were classified as a single PET. ChIA-PET analysis confirmed that upon gene activation with TNF for 30 min there were numerous PETs/contacts that occurred between the super-enhancer region and the chemokine genes, forming a putative ‘inverted rosette’ structure (FIG. 2). Further, contacts detected by ChIA-PET between interacting chemokine genes, were confined exclusively to the region encompassing the chemokine TAD. Therefore, the chemokine TAD structure may play a fundamental role in chemokine gene regulation by creating an insulated neighbourhood for transcriptional regulators, chromatin remodelers and transcribed elements. Accordingly, unraveling the mechanism whereby this TAD is regulated could reveal novel mechanisms of chemokine regulation.

A previous report demonstrated that the 5′ insulator element of the chemokine TAD engages in chromosomal contact prior to chemokine activation by TN F. Therefore, in order to investigate whether chromosomal contact between UMLILO and the chemokine was influenced by TNF treatment, we performed 3C analysis across the entire chemokine TAD. Corresponding to other innate immune genes examined in the study by Jin et al. we observed that the chemokine TAD structure is formed prior to gene activation by TNF. The transcriptional regulation of chemokines is tightly controlled, permitting chemokine expression only upon receipt of the correct signals. For this reason, in resting cells, the promoters of chemokine genes are primed to allow their swift activation and the immediate mobilization of the neutrophil response. Taken together, this suggested a relationship between pre-formed chromosomal contact and the poised nature of the chemokine genes.

UMLILO is a Super-Enhancer Resident IncRNA Transcribed within the Chemokine TAD

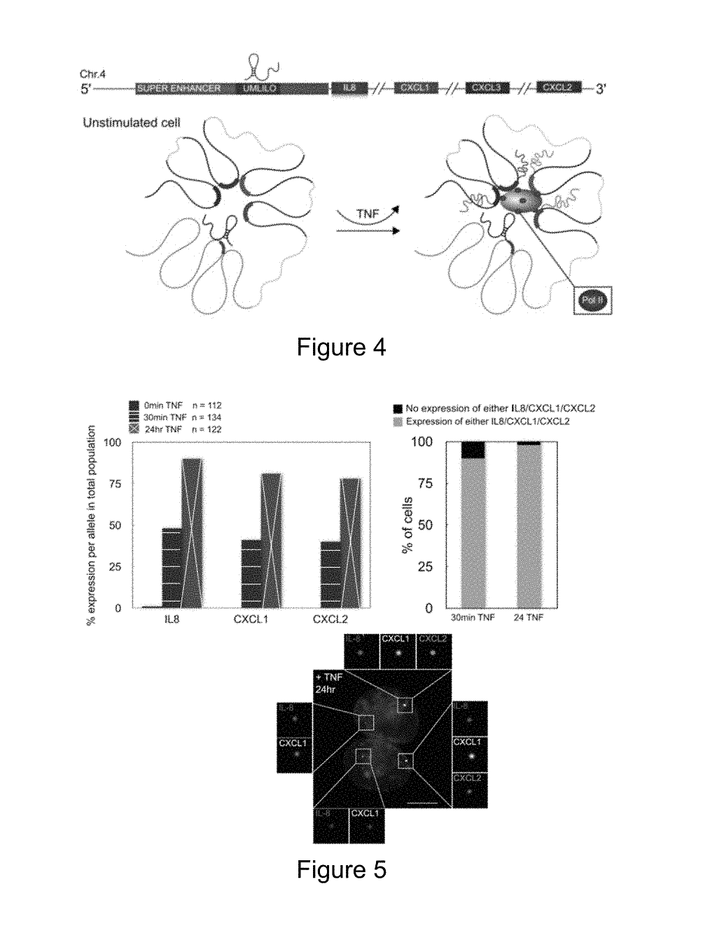

TADs containing enhancer-dense regions or super-enhancers (SE) may include co-regulated genes that are exceptionally sensitive to external stimuli, permitting fine-tuned and rapid control of gene regulation. ChIPseq analysis across the chemokine TAD revealed a previously identified SE that is atypically large (˜80 Kbp) and highly enriched for the eRNA chromatin marks, H3K4me1 and H3K27Ac (FIG. 3). This region has also been shown to be densely occupied by chromatin regulators including subunits of the Mediator complex and the chromatin reader BRD4 (Brown et al. 2014). Sites enriched for SE chromatin marks corresponded to contact regions identified by ChIA-PET (FIG. 3), suggesting that these regions may give rise to eRNA transcription. Analysis of FANTOM 5 CAGE data identified several eRNAs which corresponded to H3K4me1 and H3K27Ac peaks emanating from within the SE (FIG. 3). To investigate the existence of novel IncRNAs transcribed within the chemokine TAD, we queried the FANTOM 5 data for evidence of CAGE peaks not associated to any Entrez/GENCODE (coding) genes nor the typical eRNA chromatin marks. Our analysis allowed us to identify a transcript that was expressed from the positive strand and across numerous cell types, that we named UMLILO (Upstream Master LncRNA of the Inflammatory chemokine LOcus). This novel 575 nucleotide (nt) long transcript is a spliced and polyadenylated IncRNA that does not possess the typical eRNA histone modifications of H3K4me1 and H3K27Ac (FIG. 3). Though lacking the typical eRNA chromatin marks, ChIA-PET analysis revealed that the region encoding UMLILO formed extensive Pol II-mediated chromosomal contacts with the chemokine genes, particularly its closest chemokine neighbor, IL8 (FIG. 3). Together with the 3C data, this suggested that in unstimulated cells, UMLILO might be brought in close proximity to the chemokine genes by pre-formed chromosomal looping (FIG. 4). This data demonstrated that UMLILO is a novel IncRNA emanating from the SE, lacking typical eRNA chromatin marks, and revealed its positioning in close proximity to the chemokine genes by pre-configured nuclear architecture.

### Example 3

UMLILO Transcription Precedes Chemokine Gene Activation

The regulation of chemokine expression occurs predominantly at the transcriptional level. Historically, chemokine transcription is assayed at a population level, which lacks the resolution to reveal the exact site of active chemokine transcriptional activation. Importantly, this can only be achieved through single cell studies able to achieve single molecule resolution. Thus in order to directly observe the site of transcription at a single cell level, we designed single molecule RNA FISH (smFISH) probes to target the introns of the chemokine genes (Raj et al. 2008). As introns are typically spliced and degraded cotranscriptionally, these probes label the transcriptional start site (TSS). Of the four chemokine genes within the TAD, CXCL3 does not possess an intron and was therefore excluded from our single cell analysis experiments. Using intronic smFISH, we were able to show that the chemokines are only induced in HUVECs following TNF induction (FIG. 5). Furthermore, the RNA FISH foci of co-expressed chemokine genes always co-localized (FIG. 5). This provided supporting evidence to the Hi-C and ChIAPET data that these chemokine genes engage in chromosomal contact, and are organized into TADs. At an early time point following TNF stimulation (30 min) approximately 50% of all alleles displayed chemokine transcriptional activation. The expression frequency of the chemokine increased significantly upon stimulation with TNF for 24 hr, reaching ˜95% for IL8, ˜90% for CXCL1 and ˜85% for CXCL2 (FIG. 5). Importantly, after 30 min post TNF treatment, even though not every allele of IL8, CXCL1 and CXCL2 was expressed simultaneously, approximately 90% of cells displayed expression of either IL8, CXCL1 or CXCL2. Therefore, since all of the individual chemokines are able to induce neutrophil chemotaxis, this suggests that the transcriptional activation of at least a single chemokine per cell may be sufficient to mount a successful neutrophil response. Interestingly, this single cell perspective revealed that the expression of IL8 was not uniformly accompanied by CXCL1 and CXCL2, implicating IL8 as the dominant chemokine in this TAD. This dominance may be attributed to the proximity of IL8 to the super-enhancer, both of which are located within the same subdomain.

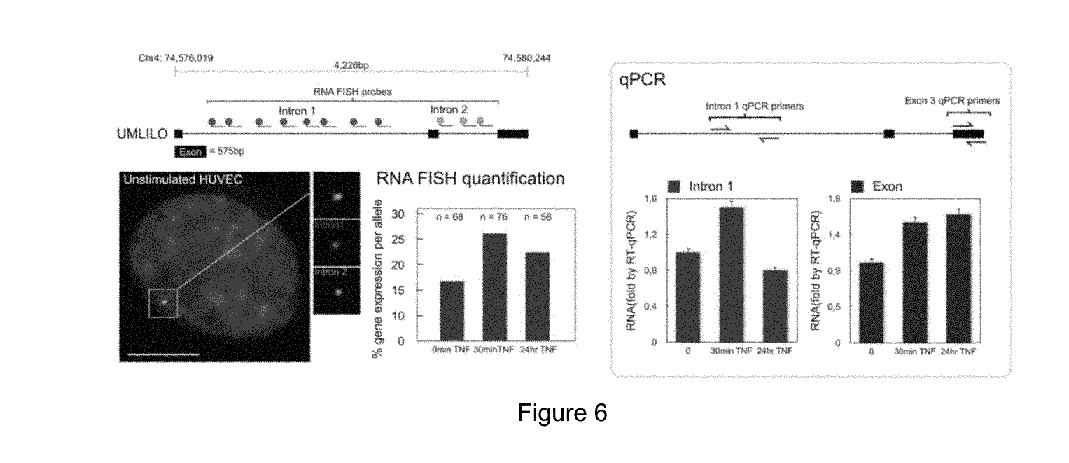

To investigate the transcriptional activation of UMLILO in HUVECs at a single cell level, we designed RNA FISH probes targeting intron 1 and intron 2 of UMLILO. RNA FISH revealed that the signal from both introns consistently colocalized (FIG. 6) at the same site of transcription. This suggests that the intronic portions of UMLILO are not redistributed to different subcellular locations. Further, intronic FISH signal was observed in both unstimulated and TNF challenged HUVECs. Although levels of intronic UMLILO were elevated shortly after TNF treatment, they dropped back down to resting UMLILO levels after longer durations of TNF stimulation (FIG. 6). This single cell RNA FISH data supported the CAGE data showing UMLILO to be transcribed in unstimulated HUVECs and prior to chemokine gene activation. In addition, we used RNA FISH to visualize the exonic portion of UMLILO. However, as the exonic form of UMLILO is only 575 nt, RNA FISH signals were difficult to detect reliably. Therefore, in order to investigate the stability of UMLILO after TNF treatment we performed qPCR on both an intronic and exonic portion of UMLILO. We observed the same pattern of intronic expression of UMLILO as we did with the RNA FISH approach. Interestingly, we observed that the exonic portion of UMLILO was slightly elevated after both 30 min and 24 hr TNF treatment. Since the level of the intronic portion of UMLILO declined 24 hr post TNF treatment, it suggests that the transcriptional activation of UMLILO is reduced at later time points after TNF treatment, but that the exonic portion of UMLILO is stabilized.

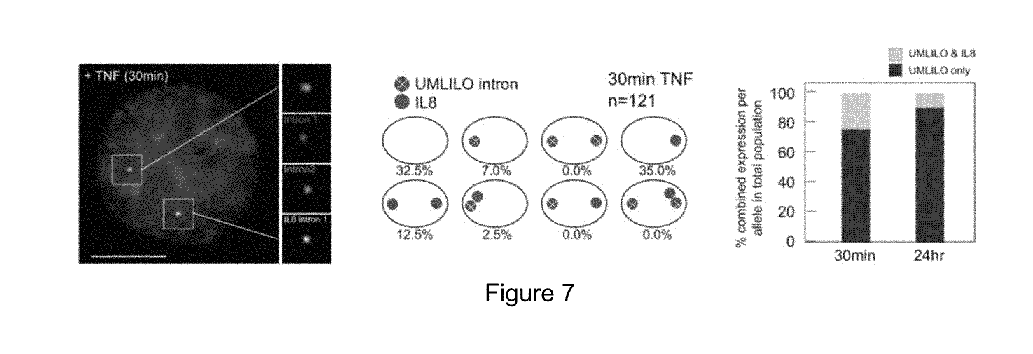

The expression of UMLILO in resting endothelial cells coincides with UMLILO being brought in close proximity to the chemokine gene promoters by pre-formed chromosomal loops (FIG. 6). However, genome-wide 3C and transcriptome approaches fail to reveal the temporal regulation of UMLILO vis-à-vis the transcriptional regulation of the chemokine genes. Therefore, we performed simultaneous intronic RNA FISH on UMLILO as well as the chemokine genes in the presence of TNF. Intronic RNA FISH revealed that overlapping co-localization between intronic UMLILO and the chemokine genes was rarely observed, suggesting that UMLILO transcription is decoupled to chemokine gene expression (FIG. 7). Interestingly, the expression of UMLILO and the chemokines was frequently observed to occur on the periphery of the nucleolus. The expression of UMLILO prior to chemokine transcriptional activation suggests a relationship between UMLILO expression and chemokine gene regulation.

UMLILO is Conserved in Higher Vertebrates, but Lacks a Homolog in Rodents

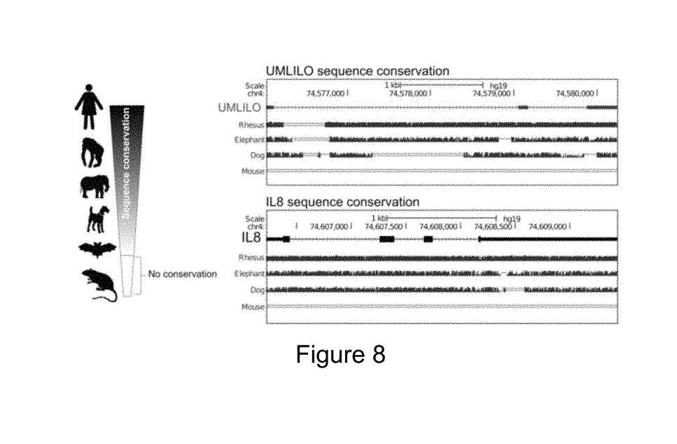

The rules governing the transcriptional regulation of IncRNAs are poorly understood, especially with respect to IncRNAs that are expressed in unstimulated cells. UMLILO was identified by CAGE to be a low abundant transcript, which is expressed in a wide variety of challenged and unchallenged immune and nonimmune cells, including primary endothelial cells. Due to the fact that most of these cell types are able to induce neutrophil chemotaxis, UMLILO may be an important regulator of the innate immune response. The chemokines comprise a critical component of the innate immune system, which in mammals is highly evolutionarily conserved. Therefore, we investigated the sequence conservation of UMLILO across metazoans. We observed that UMLILO is highly conserved in higher vertebrates, but no homolog of UMLILO exists in mice, a model used for a multitude of inflammatory disorders (FIG. 8). In addition to the absence of an UMLILO homolog, mice do not express IL8 (FIG. 8). Further, corresponding to the absence of UMLILO and IL8, mice blood has been shown to contain lower levels of neutrophils than human blood. Taken together this indicates that there may exist key differences between ELR+ CXC chemokine-dependent neutrophil chemotaxis across mammals.

### Example 4

UMLILO Transcription is Impervious to Perturbation of Super-Enhancer Transcriptional Regulators and is Necessary for Chemokine Transcription

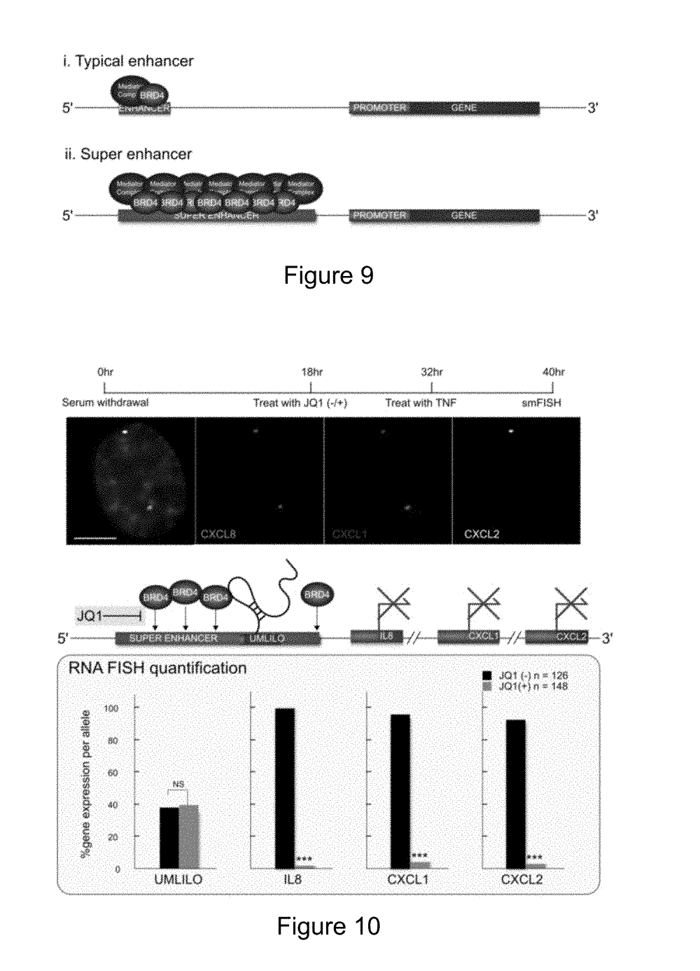

Super-enhancers (SE) are densely occupied by chromatin regulators, including the mediator subunit, MED12, and BRD4 (FIG. 9). Therefore, genes regulated by SEs have been shown to be exquisitely sensitive to depletion of either MED12 or abrogation of BRD4 chromatin occupancy by small molecule inhibitors. The expression of SE-resident IncRNAs has been shown to be higher than that of non SE-resident IncRNAs. However, it remains unknown whether the SE directly influences IncRNA transcriptional regulation. UMLILO resides within a SE that is engaged in pre-formed chromosomal contact with the chemokine genes. Therefore, we investigated whether ablating SE function would influence UMLILO expression.

Many critical proinflammatory genes, such as the chemokines, are poised prior to activation, allowing their rapid transcription upon the arrival of signal-dependent transcription factors. The promoters of the chemokine genes are enriched for histone H3 K4 mono-, di- and trimethylation (H3K4me3), which are active chromatin marks catalyzed by the MLL family of methyltransferases. These chromatin modifications permit RNA Pol II to access the promoter, where it awaits further signals to initiate active transcription. The release of “paused” Pol II is facilitated by the recruitment of the transcriptional elongation factor P-TEFb by the chromatin reader, BRD4. In the case of proinflammatory genes, various signals such as TNF or lipopolysaccharide (LPS) induce the nuclear translocation of NF-κB. The p65 subunit of NF-κB then associates directly with BRD4, prompting the release of the “paused” inflammatory genes (Kaikkonen et al. 2013). In endothelial cells, TNF induction has been demonstrated to redistribute the BRD4 payload away from resting SE, to establish new SE domains. Critically, these newly activated SEs are adjacent to canonical inflammatory genes. Therefore, we used the bromodomain inhibitor, JQ1, to investigate how the loading of the super-enhancer with BRD4/NF-κB influenced UMLILO and chemokine expression. Corresponding to a previous report, (+) JQ1 but not its enantiomer (−) JQ1 abrogated chemokine transcriptional activation (FIG. 10). Interestingly, JQ1 did not influence UMLILO transcription (FIG. 10). This supports the observation that UMLILO is expressed prior to chemokine transcription and therefore, uninfluenced by preventing the release of “paused” pol II. Further, this reveals a possible alternative regulatory mechanism for UMLILO transcription, which is independent to CXC chemokine transcriptional regulation.

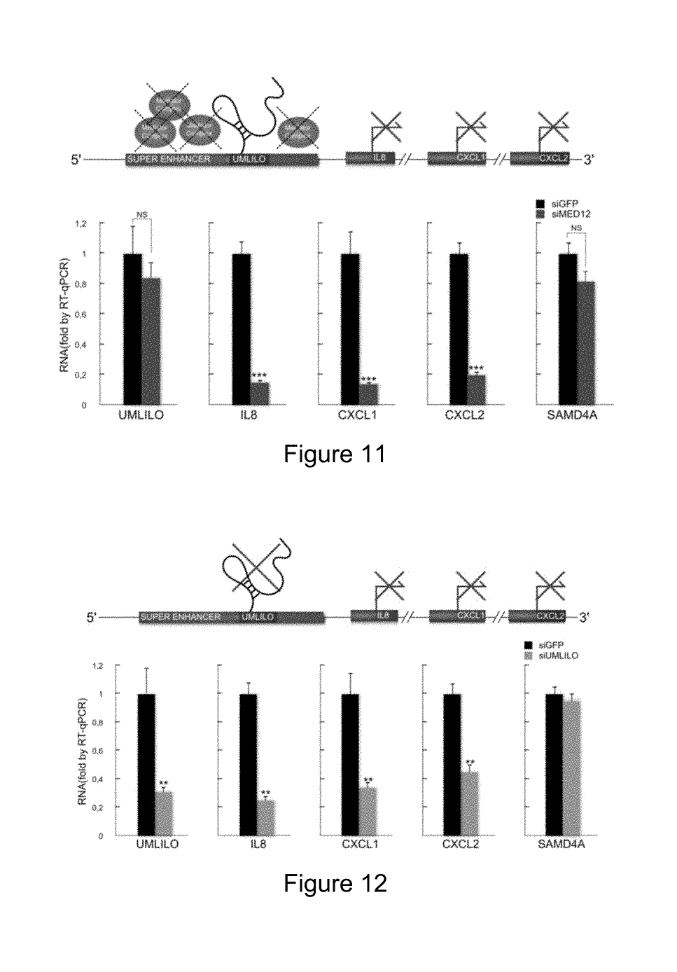

Through their interaction with MED12, eRNAs have been proposed to be the architects of chromatin organization and therefore putatively TADs. We found that depleting MED12 abrogates the expression of the CXC chemokines (FIG. 11). This suggests that, through the activity of eRNAs, the super-enhancer region in the cytokine TAD may be coordinating chemokine expression. A previous study showed that depleting subunits of the Mediator complex did not alter the expression of the activating RNA, ncRNA-a7. Similarly, knockdown of MED12 did not influence UMLILO expression (FIG. 11). Collectively, this revealed that although the super-enhancer is clearly important to chemokine transcriptional activation, it does not seem to have an impact on UMLILO transcriptional regulation.

It remains unknown whether IncRNAs arising from within super-enhancers are an artifact produced due to the elevated levels of transcriptional regulators occupying super-enhancers, or if they are functionally necessary for target gene expression. 3C-based approaches had thus far shown that the chemokine TAD assembles into an ‘inverted rosette’, with pre-formed contact occurring between the chemokines and UMLILO (FIG. 2). Further, coinciding with the pre-formed contact was the transcriptional activity of UMLILO in unstimulated cells (FIG. 6). Therefore, we sought to investigate whether the expression of UMLILO prior to chemokine activation is necessary to their transcription. We employed small interfering RNAs (siRNA) to knockdown UMLILO RNA and investigated chemokine expression by RT-qPCR. siRNA knockdown of UMLILO, in both HUVECs and HeLas, was sufficient to significantly abrogate chemokine expression, but not another TNF-induced gene located in a different TAD (FIG. 12). This revealed UMLILO transcription is not an artifact of the SE, but is indeed necessary for chemokine gene activation. In sum, this revealed that both the super-enhancer and UMLILO act as independent factors to drive chemokine transcription.

### Example 5

UMLILO Acts in Cis to Regulate Chemokine Expression

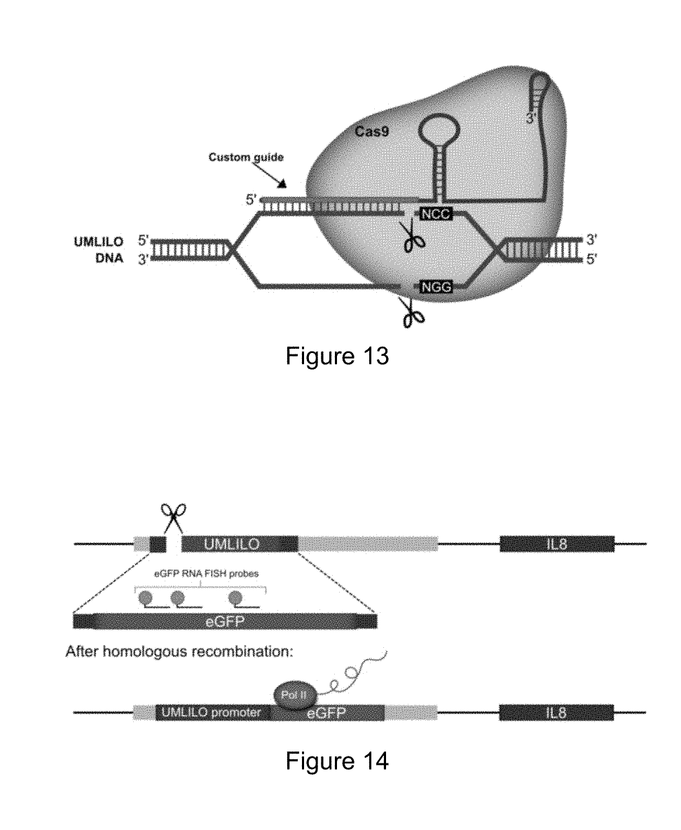

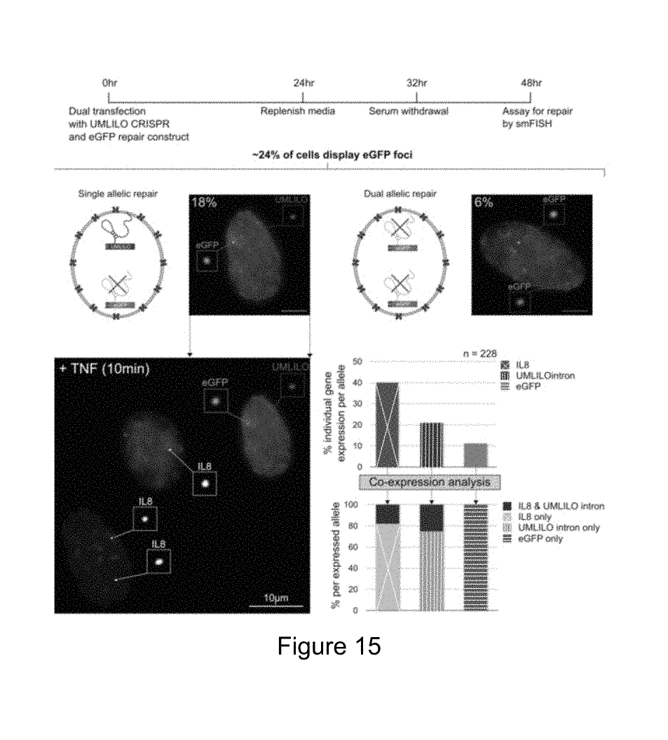

The activity of cis-acting IncRNAs appears to be dependent on these IncRNAs being transcribed at an exact genomic location. Indeed, ectopic expression of HOTTIP was insufficient to rescue the effects of HOTTIP depletion (Wang et al. 2011). siRNA approaches fail to address whether a IncRNA is acting in cis or in trans. Therefore, we devised a single cell microscopy-based approach, using the CRISPR-Cas9 system to erase the genomic sequence encoding UMLILO and replace it with an eGFP reporter sequence in primary HUVEC cells (FIG. 13). Unlike artificial tethering systems (Wang et al. 2011), this approach incorporates a critical aspect, namely the endogenous nuclear environment. Successful removal of UMLILO from its genomic locus and its replacement with the DNA sequence of eGFP was detected using RNA FISH probes targeting eGFP RNA. eGFP transcription was driven by the endogenous UMLILO promoter (FIG. 14). Following repair, we observed distinct eGFP foci in the nucleus of ˜25% of cells, with ˜18% of cells displaying a single focus, and ˜7% of cells displaying dual eGFP foci (FIG. 15). Due to the low activity of the endogenous UMLILO promoter, eGFP foci in the cytoplasm were seldom observed. Importantly cells displaying a single eGFP focus were still able to express UMLILO from the opposite allele, whereas UMLILO expression was never observed in cells displaying dual eGFP foci. Therefore, the comparison between cells displaying a single or dual eGFP foci was used as an indication of cis or trans effects of UMLILO. In agreement with siRNA data, co-localization between eGFP foci and IL8 was never observed (FIG. 15). This provided supporting evidence for the siRNA data where the silencing of UMLILO resulted in the significant downregulation of the chemokine genes.

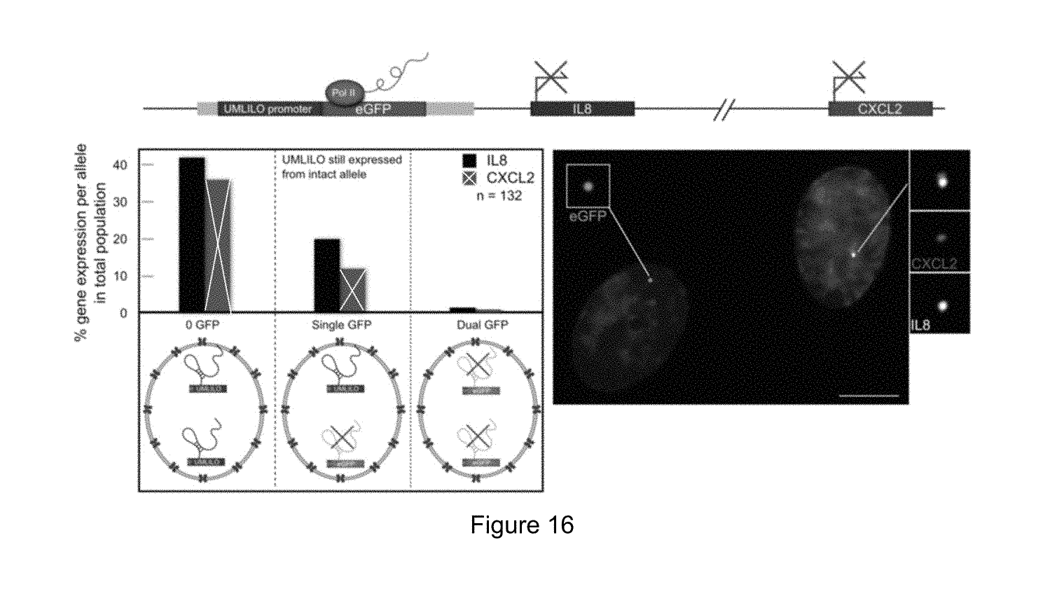

To investigate whether UMLILO exerts its effects in cis or trans, we compared cells displaying a single eGFP focus but still expressing UMLILO from the intact allele, to cells displaying dual eGFP foci. Interestingly, in cells displaying no eGFP foci, expression of IL8 and CXCL2 is comparable to the mock transfected cells (FIG. 16). However, in cells displaying a single eGFP focus, the expression of IL8 and CXCL2 was reduced significantly (FIG. 16). Importantly, this is observed despite the fact that these cells are presumed to still be able to express UMLILO from the opposite allele. This suggests that UMLILO is unable to exert its effect in trans, but rather exerts its activity from a precise genomic location in cis within the chemokine TAD. Strongly supporting the siRNA data, chemokine expression was never observed in cells displaying dual eGFP foci (FIG. 16). This definitively proves the necessity of the UMLILO transcript for the transcriptional activation of the chemokine genes.

### Example 6

UMLILO Interacts with the WAR Complex to Maintain H3K4Me3 Epigenetic Regulation of Chemokine Genes

The data thus far had shown UMLILO to be an enhancer-like cis-acting IncRNA (FIGS. 13, 14, 15 and 16) that is expressed at low abundance in unstimulated cells and brought into close proximity to target genes by chromosomal looping. As HOTTIP shares the same attributes (Wang et al. 2011), we hypothesized that UMLILO would share the same protein binding partners. HOTTIP is a IncRNA of the human HOXA locus that binds directly binds to the WDR5 protein to recruit the MLL H3K4 methylase complex to maintain H3K4me3 across the HoxA gene promoters (Wang et al. 2011). MLL1 is one of six members of the MLL family, which regulates hematopoiesis through the regulation of H3K4 methylation of the Hox genes. MLL1 has been implicated as the H3K4 methyltransferase of the innate immunity genes as it has been found to selectively regulate the activation of TNF- and LPS-induced gene activation (Wang et al. 2012). MLL1 activity is mediated via direct protein-protein interactions with the WAR complex (comprised of WDR5, Ash2L and RbBP5) as well as other regulators.

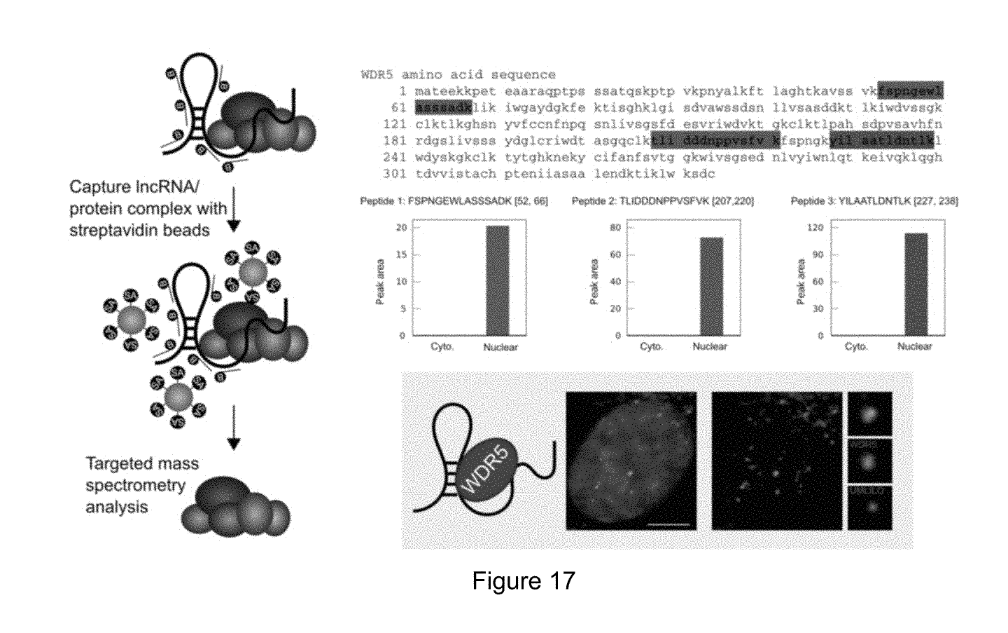

UMLILO was recovered from the nuclear fraction of cell lysates using biotinylated single stranded DNA probes tiling the exonic portion of the IncRNA. In order to verify the components of the WAR complex we used a targeted Multiple Reaction Monitoring with High Resolution Mass Spectrometry (MRMHR) method (FIG. 17). Targeted MS approaches, such as MRMHR, are generally accepted as more sensitive, specific and reproducible compared to discovery Mass Spectrometry (MS) methods. Unlike standard discovery-based approaches, MRMHR allows the quantification of proteins in the samples. Using MRMHR, we confirmed the presence of WDR5, but not Ash2L, RbBP5 or DY30, in cytoplasmic or nuclear-extracted UMLILO pull downs (FIG. 17). Consistent with previous studies demonstrating the promiscuity of RNA-binding by the C-terminal of MLL1, we detected its non-specific binding to RNA, including control IncRNA lincRNAp21 as well as UMLILO pulldowns (Wang et al. 2011). Immunofluoresence labeling revealed that WDR5 is a low abundant protein located predominantly in the nucleus in discrete punctate spots. Immuno-RNA FISH revealed that distinct foci of WDR5 colocalized with UMLILO (FIG. 17) providing supporting evidence for the UMLILO-WDR5 interaction detected by the mass spectrometry approach.

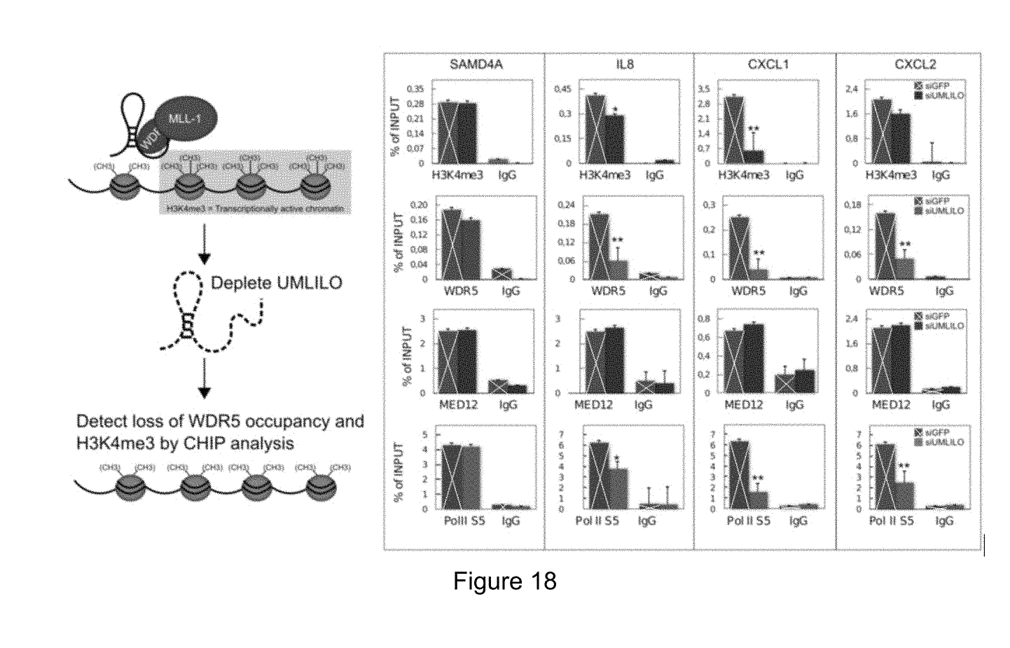

To investigate whether UMLILO was targeting the WDR5-MLL1 methylase complex to the chemokine promoters, and to interrogate the specificity of the UMLILO-WDR5 interaction, we performed siRNA knockdown of UMLILO followed by WDR5, H3K4me3, Pol II Ser5 and MED12 CHIP analysis of the chemokine promoters. Consistent with the lack of eRNA chromatin marks on UMLILO, siUMLILO did not alter MED12 occupancy on the chemokine promoters (FIG. 18). Corresponding to the mass spectrometry and IF-FISH data, we observed that siUMLILO modulates WDR5 binding as well as Pol II-Ser5 and H3K4me3 occupancy on chemokine promoters (FIG. 18). This indicated that the loss of chemokine expression observed upon UMLILO knockdown, or CRISPR-mediated deletion, is likely to be due to loss of H3K4me3 across the chemokine promoters.

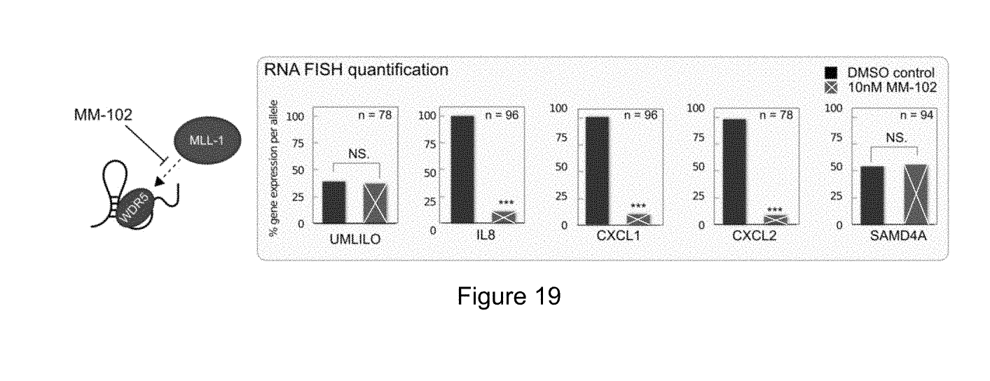

Activity of MLL1 is particularly reliant on its association with WDR5, a multifunctional adapter protein that identifies H3K4 methylation marks on the promoters of genes as well as binds to MLL1. As a consequence, small molecule inhibitors that prevent the WDR5/MLL1 interaction lead to a significant reduction in MLL1 methyltransferase activity (Cao et al. 2014). We introduced such a small molecule inhibitor, MM102, into HUVECs to prevent the WDR5/MLL1 interaction, and observed a significant reduction in chemokine gene transcription (FIG. 19). These data conclusively demonstrated the molecular mechanism by which UMLILO acts. Further, this reveals that the UMLILO-WDR5 interaction is specific and can be targeted to ablate chemokine expression through loss of H3K4me3 across chemokine gene promoters.

### Example 7

Depleting UMLILO does not Abrogate Chromosomal Contact Across the Chemokine TAD

The mechanism underpinning the pause release of Pol II on the promoters of the primed inflammatory genes is well studied. However, the upstream molecular events that coordinate the poised state of inflammatory genes, such as the chemokines, are poorly understood. Important outstanding questions include; what directs the preinitiation complex to the chemokine promoters to facilitate the methylation of the chemokine genes? And, how does 3D chromatin topology influence this regulation? In a resting state, the chemokine TAD is comprised of pre-formed chromosomal loops. Moreover, through the activity of UMLILO, the promoters of the chemokine genes are marked with active epigenetic marks prior to chemokine gene activation (FIG. 18). This suggests a relationship between the preformation of the chemokine TAD and the epigenetic activation of the chemokine genes.

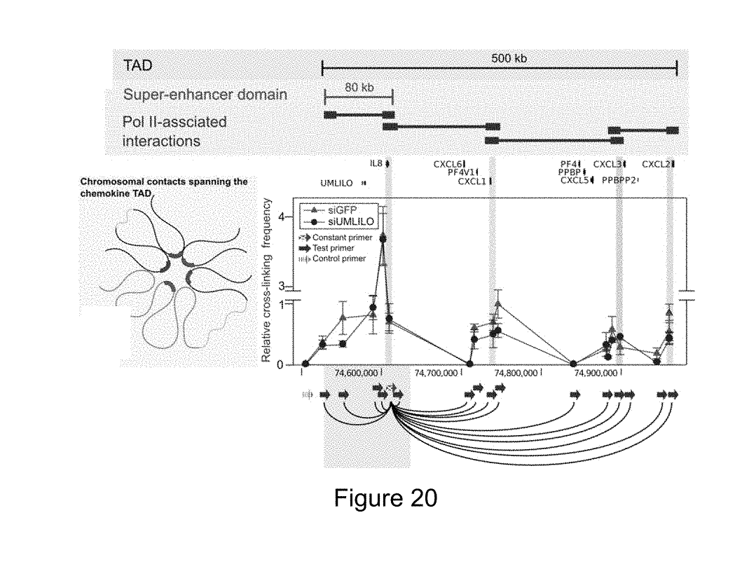

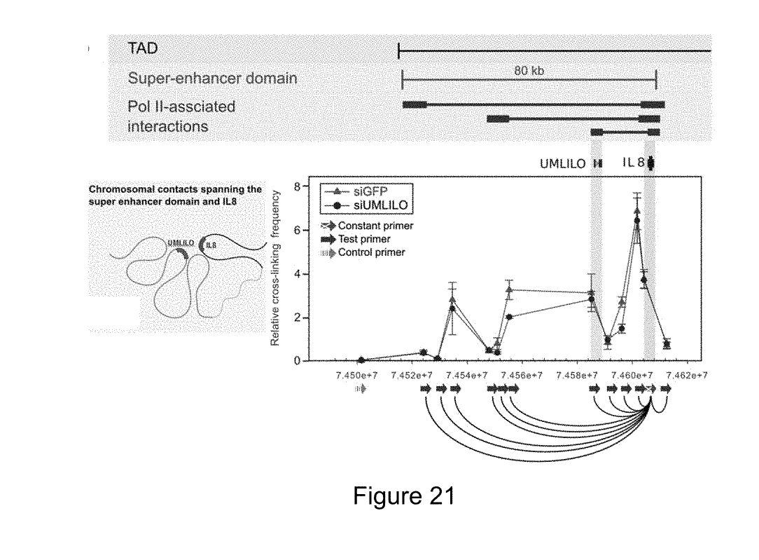

5C analysis revealed that although HOTTIP is brought in close proximity to the HoxA genes by chromosomal looping, the higher order chromatin structure of the HoxA locus remained unaltered in HOTTIP-depleted cells (Wang et al. 2011). However, this approach lacked the resolution to determine whether intra-TAD contacts were formed prior to HoxA gene activation. Therefore, we sought to investigate whether the pre-formed chromosomal contacts occurred upstream of the epigenetic activation of the chemokines. Across three biological replicates, siUMLILO followed by 3C analysis revealed that chromosomal contact across the chemokine TAD remained unaffected in the absence of UMLILO (FIG. 20). Notably, chromosomal interactions between UMLILO and IL8 remained unaltered (FIG. 21). In sum these chromatin architecture data revealed that stable, or pre-formed, chromosomal interactions in the chemokine TAD occur upstream of H3K4me3 epigenetic activation. Further, the 3D chromatin topology in the chemokine TAD contributes directly to the positioning of UMLILO in the appropriate genomic location, to allow the epigenetic activation of chemokine expression.

### Example 8

The Activity of UMLILO can be Substituted with a Different WDR5-Interacting IncRNA

Both HOTTIP and UMLILO use ‘pre-configured’ chromatin compaction to direct the WDR5-MLL1 complex in close vicinity of their target genes (FIGS. 20 and 20; Wang et al. 2011). This reveals that interactions between IncRNAs and WDR5 may be a widely used mechanism of gene activation in 3D nuclear compartments. However, to our knowledge, the interchangeability of cis-acting IncRNAs with similar molecular functions has never been functionally explored. Therefore, we hypothesized that exchanging HOTTIP with UMLILO, at its endogenous location within the chemokine TAD, may be sufficient to enable successful activation of the chemokine genes.

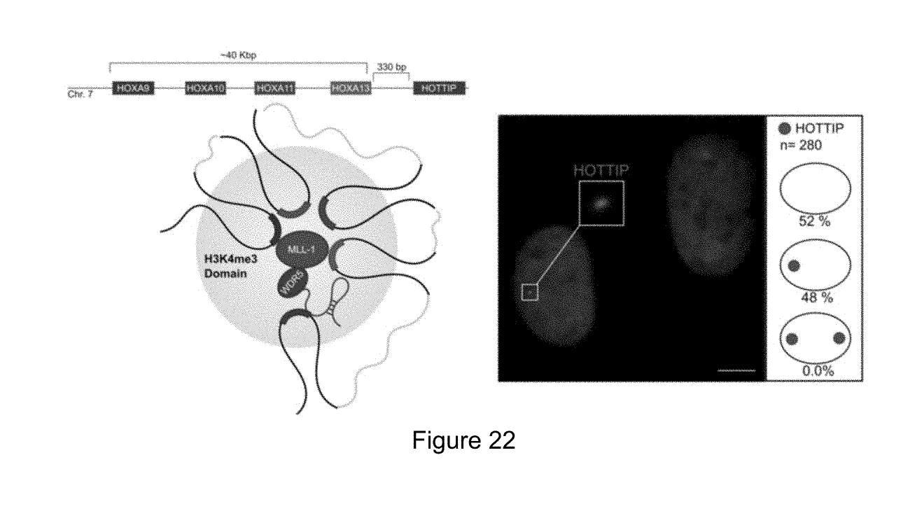

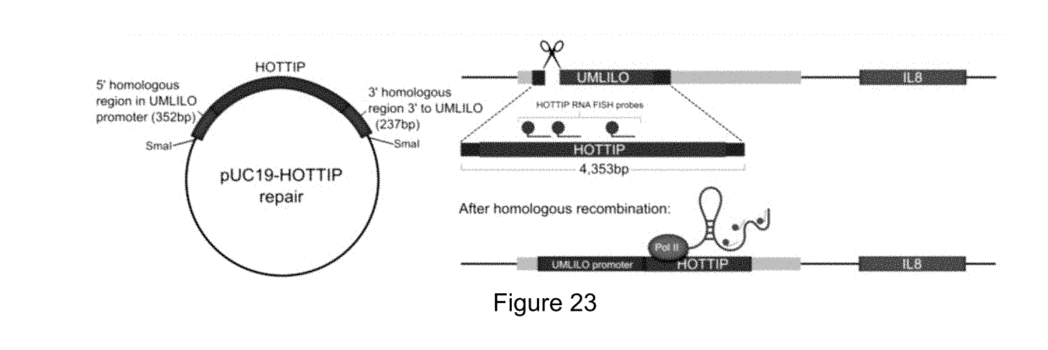

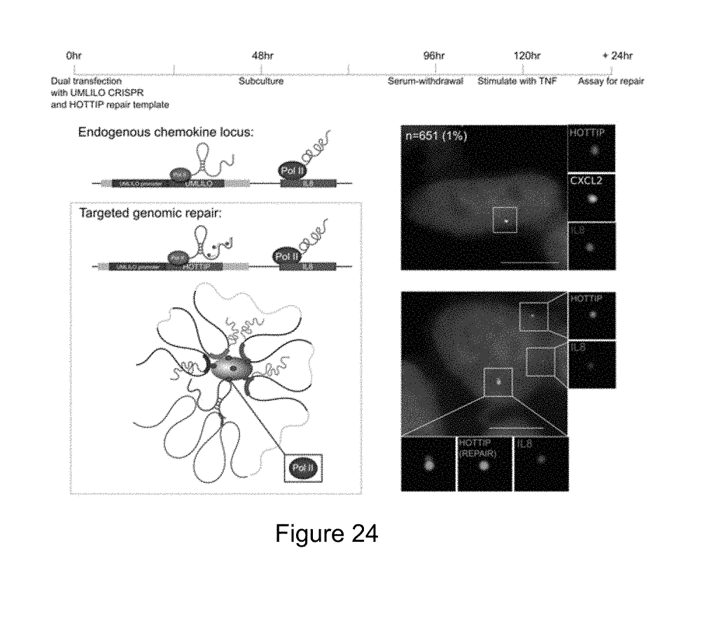

We generated a repair template that included the sequence of the HOTTIP IncRNA, with homologous arms that flanked UMLILO (FIG. 23). By transfecting cells with the repair template as well as a CRISPR that induced a double strand break near to the transcription start site of UMLILO, we aimed to exploit homologous-directed recombination (HDR) to delete the UMLILO DNA sequence, and replace it with the HOTTIP DNA sequence under the control of the endogenous UMLILO promoter. We then used TNF to induce chemokine expression in cells where the recombination event had successfully occurred. We detected the transcription of HOTTIP using RNA FISH probes that bind to its RNA and related the position of HOTTIP transcription to the chemokine genes. Due to HDR with such a long repair construct being a rare event and as we observed very poor transfection efficiency and repair in the primary HUVECs (data not shown), we performed the genome editing experiments in a well-characterized HeLa cell line. In unstimulated, serum-starved HeLas, HOTTIP FISH spots were observed at approximately 25% of alleles (FIG. 22). Importantly, in our HeLa cell line only single allelic expression of HOTTIP was ever observed in each cell. Further, co-localization between HOTTIP and the chemokines was never observed in control transfected cells. Strikingly, despite the lack of antibiotic selection, we observed overlapping HOTTIP with IL8 and/or CXCL2 foci in ˜1% of transfected cells (FIG. 24). Notably, in several instances, overlapping foci were observed in cells displaying two HOTTIP foci. Remarkably, this suggests that by exchanging IncRNAs with similar molecular functions in the appropriate genomic location, functional substitution is possible. Here we were able to restore chemokine gene activation by substituting one WDR5-binding IncRNA for another. This is, to our knowledge, the very first observation revealing the interoperability of IncRNAs with similar functions. This striking finding demonstrates that IncRNA function is dependent on both the activity encoded in their RNA structure as well as their expression in the appropriate 3D genomic context.

### DISCUSSION

Here we report UMLILO, a new super-enhancer (SE) resident enhancer-like IncRNA that interacts with WDR5 to coordinate the H3K4me3 epigenetic activation of the highly therapeutically relevant chemokine locus. Many characterized IncRNAs are identified based on their strong upregulation by external stimuli, such as LPS or DNA damage. Subsequently, low copy number IncRNAs that are expressed in resting cells and prior to stimulation receive less attention. Uniquely, UMLILO was identified using a ‘guilt-by association’ approach, by analyzing all IncRNAs transcribed across the chemokine TAD in unchallenged immune and nonimmune cells. The ubiquitous expression of UMLILO across various cell types reveals that it may be an important regulator of the innate immune response.

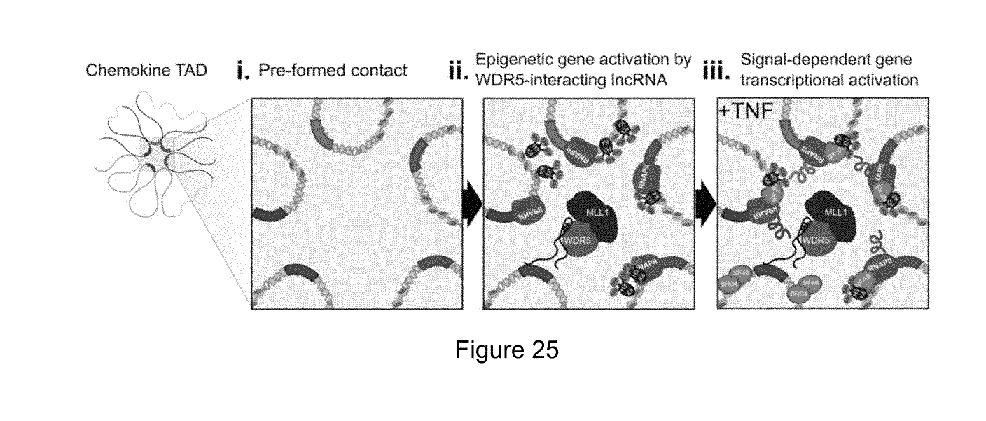

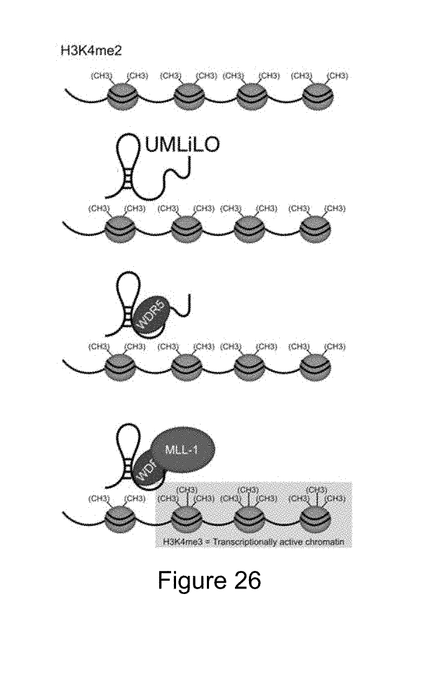

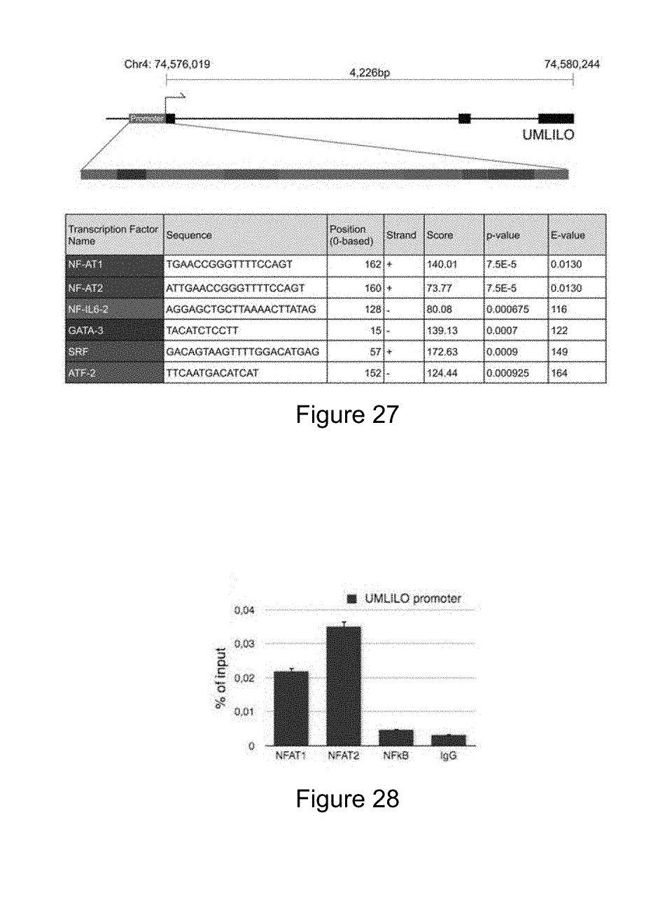

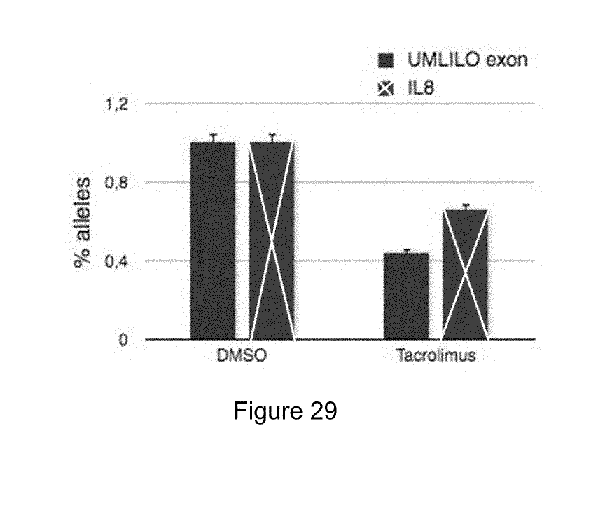

The functional relationship between 3D chromatin structure and rapidly responding genes remains opaque. Therefore, we used the chemokine TAD as a model to dissect the temporal events that are required to achieve chemokine gene expression. Chromosomal contacts across the TAD were not influenced by TNF induction of the chemokines, indicating that pre-formed chromosomal contact within the chemokine TAD forms upstream of chemokine transcription. In support of this notion, siRNA depletion of UMLILO did not alter the pre-formed chromosomal contacts demonstrating that the formation of the TAD precedes UMLILO-mediated activity (FIGS. 20, 21 and 25). Although UMLILO arises from within a SE, its transcription is decoupled from SE function (FIGS. 3, 9, 10, 11 and 12). This demonstrates that the epigenetic activation of the chemokine may proceed independently of the loading of the SE with transcriptional regulators (FIG. 25). Thus, upon activation by TNF, the chemokine SE becomes loaded with BRD4 and other transcriptional regulators, to induce the rapid and robust transcriptional induction of the chemokine genes (FIG. 25). Therefore, the sequential combination of all three events; preformation of chromosomal contact; epigenetic priming of chemokines; signal-dependent loading of transcription factors on the SE, is required to achieve chemokine transcriptional activation (FIG. 25). Intriguingly, we also show that the activity of UMLILO can be substituted with a different WDR5-interacting enhancer-like IncRNA. This reveals that the pre-configured compartment is a critical factor in constraining the location of cis-acting IncRNAs, which in turn, coordinates the activation of genes located within the same compartment. This study is to our knowledge, the first demonstration of the temporal sequence of how 3D nuclear architecture and epigenetic gene priming influence chemokine gene transcription.

Owing to the delays that occur due to the assembly of the preinitiation complex and eviction of inhibitory nucleosomes at the promoter, genes that lack poised Pol II may exhibit more stochastic patterns of gene expression. However, stochasticity in innate immune gene expression may be detrimental to mounting a successful immune response. Therefore, by maintaining the promoters of the innate immune genes in an active state, eukaryotic organisms may have evolved to reduce gene-intrinsic noise. Indeed, the “poised” innate immune genes in humans, such as the CXC chemokines, need to respond immediately to external stimuli, and therefore, exhibit a robust transcriptional response upon activation across nearly all cells (FIG. 5). Therefore, we speculate that genes displaying fast, nonstochastic gene expression exhibit a distinct TAD nuclear architecture and may also be assisted in transcriptional activation by enhancer-like IncRNAs such as UMLILO. In addition, they may be located within a TAD that may contain a SE, and engage in pre-formed chromosomal contact with similar epigenetic priming. This preformation of nuclear architecture around the SE may create a domain of IncRNAs, chromatin remodelling proteins and other transcriptional regulators in close vicinity to target genes. As a consequence, target gene promoters would overcome noise associated with stochastic promoter activation, and therefore, upon the arrival of signal-dependent transcription factors would be able to achieve rapid transcriptional activation. Contrastingly, genes that are located in a TAD lacking pre-formed contact, may be subject to dynamic loop-mediated regulation and exhibit stochastic gene expression, evoking spatially variegated gene expression patterns. This suggests that the assembly of co-regulated genes into a pre-formed “inverted rosette” may be a general feature of poised genes, such as immune genes, facilitating their rapid activation.

TNF is an established mediator of sepsis-associated inflammatory processes. Therefore, a detailed understanding of TNF-induced transcriptional responses is vital to develop new therapies that curb the excessive cytokine release that occurs during sepsis. Despite being used as a model for various inflammatory disorders, mice have been reported to be highly resistant to inflammatory stimuli when compared with humans. Indeed, early in vivo studies revealed that the lethal dose of LPS in half of the population of mice was 1,000-10,000 times the dose that is associated with septic shock and severe disease in humans. One explanation may be that mice lack several components involved in innate immune signaling: notably IL8 (FIG. 8). Furthermore, although mice do possess orthologs for CXCL1, CXCL2 and CXCL3, they possess neutrophil-poor blood when compared to higher vertebrates. A curious observation from this study is the lack of any UMLILO homolog in mice, despite its clear role in human chemokine regulation. Interestingly, despite lacking UMLILO, Cxcl2 and Cxcl5 levels were significantly altered in Mill (−/−) knockout mice (Wang et al. 2012). As NeST is a IncRNA that acts in trans to direct WDR5 and MLL1 to target IFN-γ in humans and mice (Gomez et al. 2013), we cannot rule out that a trans-acting IncRNA regulates chemokine expression in mice. However, as neutrophil chemotaxis is a critical component of innate immune signaling in higher vertebrates, we speculate that cis-acting IncRNAs may have evolved to ensure more robust chemokine expression.

Historically, upstream steps that regulate chemokine signal transduction are targeted for therapeutic intervention. Unfortunately, due to broad off-target effects, these approaches have been largely unsuccessful. More recently, agonists targeting the CXC chemokine receptors, CXCR1 and CXCR2, are thought to be a viable option to attenuate chemokine signalling. However, there is concern that these global approaches may lead to immunosuppression upon extended treatment. Therefore, there is a great need to develop highly specific small molecule inhibitors that are able to more discretely influence chemokine transcriptional regulation. As UMLILO is essential to the epigenetic activation of the chemokines, it represents a novel approach to drug the chemokine transcriptional response. Indeed, a small molecule inhibitor that inhibits the WDR5/MLL1 interaction led to a significant reduction in chemokine expression (FIG. 19). As aberrant expression of these chemokines and other cytokines underlies multiple disease states, adjustment of chemokine levels by altering the activity of UMLILO, may represent a valuable therapeutic strategy.

### REFERENCES

Andersson, R, et al. (2014). An atlas of active enhancers across human cell types and tissues. Nature 507, 455-461.
Cao, F, et al. (2014). Targeting MLL1 H3K4 methyltransferase activity in mixed-lineage leukemia. Mol Cell. 53, 247-261.
Cong, L, et al. (2013). Multiplex genome engineering using CRISPR/Cas systems. Science 339, 819-23.
Grebien, F, et al. (2015). Pharmacological targeting of the Wdr5-MLL interaction in C/EBPα N-terminal leukemia. Nat Chem Biol. 11, 571-8.
Hagege, H, et al. (2007). Quantitative analysis of chromosome conformation capture assays (3C-qPCR). Nat. Protoc. 2, 1722-1733.
Gomez, J A, et al. (2013). The NeST long ncRNA controls microbial susceptibility and epigenetic activation of the interferon-g locus. Cell 152, 743-754.
Jin, F. (2013). A high-resolution map of the three-dimensional chromatin interactome in human cells. Nature 503, 290-294.
Li, G, et al. (2010). ChIA-PET tool for comprehensive chromatin interaction analysis with paired-end tag sequencing. Genome Biol. 11, R22.
Papantonis, A, et al. (2012). TNFα signals through specialized factories where responsive coding and miRNA genes are transcribed. EMBO J. 31, 4404-4414.
Paulsen, M T, et al. (2013). Coordinated regulation of synthesis and stability of RNA during the acute TNF-induced proinflammatory response. Proc Natl Acad Sci USA. 110, 2240-5.
Raj, A, et al. (2008). Imaging individual mRNA molecules using multiple singly labeled probes. Nat. Methods 5, 877-879.
Shevchenko, A, et al. (2007). In-gel digestion for mass spectrometric characterization of proteins and proteomes. Nature Protocols. 1, 2856-2860.
Tennakoon, C, et al. (2012). BatMis: a fast algorithm for k-mismatch mapping. Bioinformatics 28, 2122-8.
Wang, K C, et al. (2011). A long non-coding RNA maintains active chromatin to coordinate homeotic gene expression. Nature 472, 120-124.
Wang, X, et al. (2012). MLL1, a H3K4 methyltransferase, regulates the TNFα-stimulated activation of genes downstream of NF-κB. J Cell Sci. 125, 4058-4066.

1. A pharmaceutical composition comprising a nucleic acid comprising at least one polynucleotide selected from the group consisting of SEQ ID NO: 54, the complementary sequence of SEQ ID NO: 54, SEQ ID NO: 55, the complementary sequence of SEQ ID NO: 55, SEQ ID NO: 56, the complementary sequence of SEQ ID NO: 56, SEQ ID NO: 57, and the complementary sequence of SEQ ID NO: 57.

2. The pharmaceutical composition of claim 1, wherein the nucleic acid further comprises a promoter operably linked to the at least one polynucleotide.

3. The pharmaceutical composition of claim 1, wherein the nucleic acid comprises SEQ ID NO: 54.

4. The pharmaceutical composition of claim 1, wherein the nucleic acid comprises the complementary sequence of SEQ ID NO: 54.

5. The pharmaceutical composition of claim 1, wherein the nucleic acid comprises SEQ ID NO: 55.

6. The pharmaceutical composition of claim 1, wherein the nucleic acid comprises the complementary sequence of SEQ ID NO: 55.

7. The pharmaceutical composition of claim 1, wherein the nucleic acid comprises SEQ ID NO: 56.

8. The pharmaceutical composition of claim 1, wherein the nucleic acid comprises the complementary sequence of SEQ ID NO: 56.

9. The pharmaceutical composition of claim 1, wherein the nucleic acid comprises SEQ ID NO: 57.

10. The pharmaceutical composition of claim 1, wherein the nucleic acid comprises the complementary sequence of SEQ ID NO: 57.

11. A pharmaceutical composition comprising a viral vector comprising at least one polynucleotide selected from the group consisting of SEQ ID NO: 54, the complementary sequence of SEQ ID NO: 54, SEQ ID NO: 55, the complementary sequence of SEQ ID NO: 55, SEQ ID NO: 56, the complementary sequence of SEQ ID NO: 56, SEQ ID NO: 57, and the complementary sequence of SEQ ID NO: 57.

12. The pharmaceutical composition of claim 11, wherein the viral vector further comprises a promoter operably linked to the at least one polynucleotide.

13. The pharmaceutical composition of claim 11, wherein the viral vector comprises SEQ ID NO: 54.

14. The pharmaceutical composition of claim 11, wherein the viral vector comprises the complementary sequence of SEQ ID NO: 54.

15. The pharmaceutical composition of claim 11, wherein the viral vector comprises SEQ ID NO: 55.

16. The pharmaceutical composition of claim 11, wherein the viral vector comprises the complementary sequence of SEQ ID NO: 55.

17. The pharmaceutical composition of claim 11, wherein the viral vector comprises SEQ ID NO: 56.

18. The pharmaceutical composition of claim 11, wherein the viral vector comprises the complementary sequence of SEQ ID NO: 56.

19. The pharmaceutical composition of claim 11, wherein the viral vector comprises SEQ ID NO: 57.

20. The pharmaceutical composition of claim 11, wherein the viral vector comprises the complementary sequence of SEQ ID NO: 57.


SEQUENCE LISTING (314 sequences)

SEQ ID NO: 1 - DNA, 575 bp, Homo sapiens
atacatgtggagattaagacccataataacaatgacaacactttcataacagttcatctgtgttaacatacaaattctcgcagcaacactccagggcgctttatgtgtggatcttttttagtctgcatattaaccctacaagttggaaatggctcctctcaaacactggagatagagcagcccaaatgtatctgctactgtggtgccttccataatgcaaaactctctgaggagctgagaatatgtctactgctaccaaaattgtaacccccatcatctagtaaagagttggtacacggtgaacatttgctgtgggaatgtattctgcttcattccagaggcctgccaattcttaatctcactataggctgaagagctgctcacatagaatacttgtagtgacttccattttcaccagtttagatcagtggacagagagatgctgaattactgctcaagaagtatagatccacatgccttcaacttcagaatcttaaattagaggcgaatgttgagtctactaaactgtatagtctgtaaaggcaggaactgtatttatctcagtcatatttaat

SEQ ID NO: 2 - DNA, 20 bp, Artificial sequence
  UMLILO intron 1 qPCR forward primer
ggaaggaggggaacatggaa

SEQ ID NO: 3 - DNA, 20 bp, Artificial sequence
  UMLILO intron 1 qPCR reverse primer
ctacctgactccctccctct

SEQ ID NO: 4 - DNA, 21 bp, Artificial sequence
  UMLILO exon 3 qPCR forward primer
ggtacacggtgaacatttgct

SEQ ID NO: 5 - DNA, 21 bp, Artificial sequence
  UMLILO exon 3 qPCR reverse primer
cagcatctctctgtccactga

SEQ ID NO: 6 - DNA, 20 bp, Artificial sequence
  IL8 qPCR forward primer
agggccaagagaatatccga

SEQ ID NO: 7 - DNA, 20 bp, Artificial sequence
  IL8 qPCR reverse primer
ggacttgtggatcctggcta

SEQ ID NO: 8 - DNA, 20 bp, Artificial sequence
  CXCL1 gene qPCR forward primer
agcttgcctcaatcctgcat

SEQ ID NO: 9 - DNA, 20 bp, Artificial sequence
  CXCL1 gene qPCR reverse primer
cctcctcccttctggtcagt

SEQ ID NO: 10 - DNA, 24 bp, Artificial sequence
  CXCL2 gene qPCR forward primer
cacagtgtgtggtcaacatttctc

SEQ ID NO: 11 - DNA, 23 bp, Artificial sequence
  CXCL2 gene qPCR reverse primer
acacagagggaaacactgcataa

SEQ ID NO: 12 - DNA, 41 bp, Artificial sequence
  Repair construct forward primer
ttgaaccgggttttccagtcacatatggtgagcaagggcga

SEQ ID NO: 13 - DNA, 38 bp, Artificial sequence
  Repair construct reverse primer
actatgaagactcttgggtcacttgtacagctcgtcca

SEQ ID NO: 14 - DNA, 20 bp, Artificial sequence
  ChIP IL8 forward primer
tgggccatcagttgcaaatc

SEQ ID NO: 15 - DNA, 20 bp, Artificial sequence
  ChIP IL8 reverse primer
agtgagatggttccttccgg

SEQ ID NO: 16 - DNA, 20 bp, Artificial sequence
  ChIP CXCL1 forward primer
acgtgggtctaagggatctg

SEQ ID NO: 17 - DNA, 20 bp, Artificial sequence
  ChIP CXCL1 reverse primer
gggtctgactgtcttgcgta

SEQ ID NO: 18 - DNA, 20 bp, Artificial sequence
  ChIP CXCL2 forward primer
ctgtggtggttctcagggat

SEQ ID NO: 19 - DNA, 20 bp, Artificial sequence
  ChIP CXCL2 reverse primer
tggactctgagactctggga

SEQ ID NO: 20 - DNA, 20 bp, Artificial sequence
  ChIP CXCL3 forward primer
tctggaatccgagacgatgg

SEQ ID NO: 21 - DNA, 20 bp, Artificial sequence
  ChIP CXCL3 reverse primer
gacaggaaaggcacgacttc

SEQ ID NO: 22 - DNA, 20 bp, Artificial sequence
  ChIP SAMD4A forward primer
gagctttgggtggagagagt

SEQ ID NO: 23 - DNA, 20 bp, Artificial sequence
  ChIP SAMD4A reverse primer
ttactcctcctcctcctccc

SEQ ID NO: 24 - DNA, 22 bp, Artificial sequence
  ChIP UMLILO forward primer
tgtccaaatccacattgacagt

SEQ ID NO: 25 - DNA, 20 bp, Artificial sequence
  ChIP UMLILO reverse primer
ggagtgttgctgcgagaatt

SEQ ID NO: 26 - DNA, 30 bp, Artificial sequence
  3C anchor
tcctctgacataatgaaaagatgagggtgc

SEQ ID NO: 27 - DNA, 30 bp, Artificial sequence
  3C primer 1
aaaatagaaaccctgaatgtaccggtaaca

SEQ ID NO: 28 - DNA, 30 bp, Artificial sequence
  3C primer 2
tcaaatccgtgatcagcattaccaagccat

SEQ ID NO: 29 - DNA, 28 bp, Artificial sequence
  3C primer 3
attggaaggctaaatatacttacatggc

SEQ ID NO: 30 - DNA, 27 bp, Artificial sequence
  3C primer 4
ttttaaaagcagcactagtgtatccgg

SEQ ID NO: 31 - DNA, 30 bp, Artificial sequence
  3C primer 5
aaagagctcaatgtgtgttactaaagaatg

SEQ ID NO: 32 - DNA, 29 bp, Artificial sequence
  3C primer 6
aaaggtaagcaataattggcccatatctc

SEQ ID NO: 33 - DNA, 30 bp, Artificial sequence
  3C primer 7
aaaagtgatacatgttcattgcatttaaac

SEQ ID NO: 34 - DNA, 26 bp, Artificial sequence
  3C primer 8
cgtaaggctggtcaaggtatgctgag

SEQ ID NO: 35 - DNA, 30 bp, Artificial sequence
  3C primer 9
ttcttcattatgctagtgttgtgtattgtg

SEQ ID NO: 36 - DNA, 30 bp, Artificial sequence
  3C primer 10
tgaaattagagaaaaacatgtacttaggga

SEQ ID NO: 37 - DNA, 26 bp, Artificial sequence
  3C primer 11
caccccgcccatttgatgaactgttt

SEQ ID NO: 38 - DNA, 28 bp, Artificial sequence
  3C primer 12
acttcaaactccaaactccaccgatttg

SEQ ID NO: 39 - DNA, 30 bp, Artificial sequence
  3C primer 13
ccaacatcactgaagcaaagaaacttggag

SEQ ID NO: 40 - DNA, 26 bp, Artificial sequence
  3C primer 14
tctgcttgtacgtaggtatgtagatt

SEQ ID NO: 41 - DNA, 30 bp, Artificial sequence
  3C primer 15
gctagcaaccaactctttaagaatacagcc

SEQ ID NO: 42 - DNA, 25 bp, Artificial sequence
  3C primer 16
agaaacacaacgatacaatgtgaaa

SEQ ID NO: 43 - DNA, 29 bp, Artificial sequence
  3C primer 17
agtgtgactccaaataacctagtttgcta

SEQ ID NO: 44 - DNA, 26 bp, Artificial sequence
  3C primer 18
acacagtcataatcacaaccccagtc

SEQ ID NO: 45 - DNA, 25 bp, Artificial sequence
  3C primer 19
tcagactcatgggctcagttgattc

SEQ ID NO: 46 - DNA, 30 bp, Artificial sequence
  3C primer 20
ggctgacacattatggtctcccactaaata

SEQ ID NO: 47 - DNA, 28 bp, Artificial sequence
  3C primer 21
gaaacatgtcaagaggccgtggacattt

SEQ ID NO: 48 - DNA, 26 bp, Artificial sequence
  3C primer 22
agtgtgaaacaaaacgagaagggaag

SEQ ID NO: 49 - DNA, 30 bp, Artificial sequence
  3C primer 23
aaattatttgctttaggaagggaagtagaa

SEQ ID NO: 50 - DNA, 30 bp, Artificial sequence
  3C primer 24
cataggagagactgcgacagaaattccatt

SEQ ID NO: 51 - DNA, 30 bp, Artificial sequence
  3C primer 25
ttacaactcctacaaccgtgcttggtacat

SEQ ID NO: 52 - DNA, 24 bp, Artificial sequence
  3C GAPDH control F1
tgccaatctccttgttttctaatg

SEQ ID NO: 53 - DNA, 23 bp, Artificial sequence
  3C GAPDH control F2
tattcccccaggtttacatgttc

SEQ ID NO: 54 - RNA, 21 nt, Artificial sequence
  UMLILO siRNA 1
aucuuaaauuagaggcgaauu

SEQ ID NO: 55 - RNA, 21 nt, Artificial sequence
  UMLILO siRNA 2
cauacaaauucucgcagcauu

SEQ ID NO: 56 - RNA, 21 nt, Artificial sequence
  UMLILO siRNA 3
aagaguugguacacggugauu

SEQ ID NO: 57 - RNA, 21 nt, Artificial sequence
  UMLILO siRNA 4
gcauauuaacccuacaaguuu

SEQ ID NO: 58 - DNA, 20 bp, Artificial sequence
  IL-8 smFISH probe 1
ttgtcctagaagcttgtgtg

SEQ ID NO: 59 - DNA, 20 bp, Artificial sequence
  IL-8 smFISH probe 2
cacagtgagatggttccttc

SEQ ID NO: 60 - DNA, 20 bp, Artificial sequence
  IL-8 smFISH probe 3
cagcttggaagtcatgttta

SEQ ID NO: 61 - DNA, 20 bp, Artificial sequence
  IL-8 smFISH probe 4
gaaatcaggaaggctgccaa

SEQ ID NO: 62 - DNA, 20 bp, Artificial sequence
  IL-8 smFISH probe 5
cttaccttcacacagagctg

SEQ ID NO: 63 - DNA, 20 bp, Artificial sequence
  IL-8 smFISH probe 6
acgctgtaggtcagaaagat

SEQ ID NO: 64 - DNA, 20 bp, Artificial sequence
  IL-8 smFISH probe 7
ggatcacatttagacatagg

SEQ ID NO: 65 - DNA, 20 bp, Artificial sequence
  IL-8 smFISH probe 8
caagaatagctttgctatct

SEQ ID NO: 66 - DNA, 20 bp, Artificial sequence
  IL-8 smFISH probe 9
aggatgtttgttaccaaagc

SEQ ID NO: 67 - DNA, 20 bp, Artificial sequence
  IL-8 smFISH probe 10
ctttatactttcctaagtgt

SEQ ID NO: 68 - DNA, 20 bp, Artificial sequence
  IL-8 smFISH probe 11
atatatgcatgctacatggt

SEQ ID NO: 69 - DNA, 20 bp, Artificial sequence
  IL-8 smFISH probe 12
tataggtaaccgtggttctc

SEQ ID NO: 70 - DNA, 20 bp, Artificial sequence
  IL-8 smFISH probe 13
ccttgctcaactcaatatta

SEQ ID NO: 71 - DNA, 20 bp, Artificial sequence
  IL-8 smFISH probe 14
aggagtggaattgtctgagt

SEQ ID NO: 72 - DNA, 20 bp, Artificial sequence
  IL-8 smFISH probe 15
gacaaatctgaggcttgtca

SEQ ID NO: 73 - DNA, 20 bp, Artificial sequence
  IL-8 smFISH probe 16
gggtatctttaaaccagaca

SEQ ID NO: 74 - DNA, 20 bp, Artificial sequence
  IL-8 smFISH probe 17
aagttatacatgcctggtct

SEQ ID NO: 75 - DNA, 20 bp, Artificial sequence
  IL-8 smFISH probe 18
gcatagaaattgccttcaga

SEQ ID NO: 76 - DNA, 20 bp, Artificial sequence
  IL-8 smFISH probe 19
gtaggcaagctaagactctc

SEQ ID NO: 77 - DNA, 20 bp, Artificial sequence
  IL-8 smFISH probe 20
gtgataccacagtgttattt

SEQ ID NO: 78 - DNA, 20 bp, Artificial sequence
  IL-8 smFISH probe 21
ggtcaatattgcataatcct

SEQ ID NO: 79 - DNA, 20 bp, Artificial sequence
  IL-8 smFISH probe 22
tcaacatcttcatggtattt

SEQ ID NO: 80 - DNA, 20 bp, Artificial sequence
  IL-8 smFISH probe 23
gttagagttctttttgtaca

SEQ ID NO: 81 - DNA, 20 bp, Artificial sequence
  IL-8 smFISH probe 24
acaacattgaacgacttcct

SEQ ID NO: 82 - DNA, 20 bp, Artificial sequence
  IL-8 smFISH probe 25
cctcagttagttctttgttt

SEQ ID NO: 83 - DNA, 20 bp, Artificial sequence
  IL-8 smFISH probe 26
tgaatattctcctagccctt

SEQ ID NO: 84 - DNA, 20 bp, Artificial sequence
  IL-8 smFISH probe 27
catgtttctagtgaactcat

SEQ ID NO: 85 - DNA, 20 bp, Artificial sequence
  IL-8 smFISH probe 28
atttggagactatggaaggc

SEQ ID NO: 86 - DNA, 20 bp, Artificial sequence
  IL-8 smFISH probe 29
ctacttcctttctaattcca

SEQ ID NO: 87 - DNA, 20 bp, Artificial sequence
  IL-8 smFISH probe 30
tttatcaacaggcacagctc

SEQ ID NO: 88 - DNA, 20 bp, Artificial sequence
  IL-8 smFISH probe 31
cacctgttgggggaaaaagt

SEQ ID NO: 89 - DNA, 20 bp, Artificial sequence
  IL-8 smFISH probe 32
ttagcactccttggcaaaac

SEQ ID NO: 90 - DNA, 20 bp, Artificial sequence
  IL-8 smFISH probe 33
gtctttatgcactgacatct

SEQ ID NO: 91 - DNA, 20 bp, Artificial sequence
  IL-8 smFISH probe 34
tttggggtggaaaggtttgg

SEQ ID NO: 92 - DNA, 20 bp, Artificial sequence
  IL-8 smFISH probe 35
tctcaatcactctcagttct

SEQ ID NO: 93 - DNA, 20 bp, Artificial sequence
  IL-8 smFISH probe 36
cataatttctgtgttggcgc

SEQ ID NO: 94 - DNA, 20 bp, Artificial sequence
  IL-8 smFISH probe 37
ttccaccttccttaatttta

SEQ ID NO: 95 - DNA, 20 bp, Artificial sequence
  IL-8 smFISH probe 95
cacctggaactttcctaaat

SEQ ID NO: 96 - DNA, 20 bp, Artificial sequence
  IL-8 smFISH probe 39
tcatttactactgtaatcct

SEQ ID NO: 97 - DNA, 20 bp, Artificial sequence
  IL-8 smFISH probe 40
tagtagttgtggaagctacc

SEQ ID NO: 98 - DNA, 20 bp, Artificial sequence
  IL-8 smFISH probe 41
aggcataaagtctaagtcca

SEQ ID NO: 99 - DNA, 20 bp, Artificial sequence
  IL-8 smFISH probe 42
gcattcaaatcatgattcct

SEQ ID NO: 100 - DNA, 20 bp, Artificial sequence
  IL-8 smFISH probe 43
tctcttccatcagaaagctt

SEQ ID NO: 101 - DNA, 20 bp, Artificial sequence
  IL-8 smFISH probe 44
aaaacttctccacaaccctc

SEQ ID NO: 102 - DNA, 20 bp, Artificial sequence
  CCL20 smFISH probe 1
aagcagccaggagcaaactc

SEQ ID NO: 103 - DNA, 20 bp, Artificial sequence
  CCL20 smFISH probe 2
tggagtagcagcactgacat

SEQ ID NO: 104 - DNA, 20 bp, Artificial sequence
  CCL20 smFISH probe 3
ggaaagagcgacacttacct

SEQ ID NO: 105 - DNA, 20 bp, Artificial sequence
  CCL20 smFISH probe 4
atagtctttcttctgtgcta

SEQ ID NO: 106 - DNA, 20 bp, Artificial sequence
  CCL20 smFISH probe 5
cagttgagctcaaggctaaa

SEQ ID NO: 107 - DNA, 20 bp, Artificial sequence
  CCL20 smFISH probe 6
taacagttttccatccctta

SEQ ID NO: 108 - DNA, 20 bp, Artificial sequence
  CCL20 smFISH probe 7
tcactacctcttgtatactt

SEQ ID NO: 109 - DNA, 20 bp, Artificial sequence
  CCL20 smFISH probe 8
caccatgggcagacaacttc

SEQ ID NO: 110 - DNA, 20 bp, Artificial sequence
  CCL20 smFISH probe 9
cagcagtaactcttctctcc

SEQ ID NO: 111 - DNA, 20 bp, Artificial sequence
  CCL20 smFISH probe 10
gcagagaggaaagcctgatc

SEQ ID NO: 112 - DNA, 20 bp, Artificial sequence
  CCL20 smFISH probe 11
cccaaatctgctgagattga

SEQ ID NO: 113 - DNA, 20 bp, Artificial sequence
  CCL20 smFISH probe 12
tagggatctctaagagccca

SEQ ID NO: 114 - DNA, 20 bp, Artificial sequence
  CCL20 smFISH probe 13
actttggctttatcctgaag

SEQ ID NO: 115 - DNA, 20 bp, Artificial sequence
  CCL20 smFISH probe 14
ttatatcactcccagaaaca

SEQ ID NO: 116 - DNA, 20 bp, Artificial sequence
  CCL20 smFISH probe 15
tctactggtaacattacaca

SEQ ID NO: 117 - DNA, 20 bp, Artificial sequence
  CCL20 smFISH probe 16
tctacatttcaaaatggccc

SEQ ID NO: 118 - DNA, 20 bp, Artificial sequence
  CCL20 smFISH probe 17
agttggggacacaggaggat

SEQ ID NO: 119 - DNA, 20 bp, Artificial sequence
  CCL20 smFISH probe 18
ctattaagttgggaggtacg

SEQ ID NO: 120 - DNA, 20 bp, Artificial sequence
  CCL20 smFISH probe 19
gggatgttgatgtcaggtat

SEQ ID NO: 121 - DNA, 20 bp, Artificial sequence
  CCL20 smFISH probe 20
aaagttggcaagcaatgtcc

SEQ ID NO: 122 - DNA, 20 bp, Artificial sequence
  CCL20 smFISH probe 21
acatgctgtaaggtaaggga

SEQ ID NO: 123 - DNA, 20 bp, Artificial sequence
  CCL20 smFISH probe 22
aatacttggacattcccgag

SEQ ID NO: 124 - DNA, 20 bp, Artificial sequence
  CCL20 smFISH probe 23
gtgttgtgtgactattgagc

SEQ ID NO: 125 - DNA, 20 bp, Artificial sequence
  CCL20 smFISH probe 24
gcatttttaggaatgcacct

SEQ ID NO: 126 - DNA, 20 bp, Artificial sequence
  CCL20 smFISH probe 25
ctggctactaagtattgaga

SEQ ID NO: 127 - DNA, 20 bp, Artificial sequence
  CCL20 smFISH probe 26
tgcttgaaggaacccttatg

SEQ ID NO: 128 - DNA, 20 bp, Artificial sequence
  CCL20 smFISH probe 27
ttcaagaatatgagcagccc

SEQ ID NO: 129 - DNA, 20 bp, Artificial sequence
  CCL20 smFISH probe 28
gtgggatatcttcagtaaga

SEQ ID NO: 130 - DNA, 20 bp, Artificial sequence
  CCL20 smFISH probe 29
gagaaacacaggacttctca

SEQ ID NO: 131 - DNA, 20 bp, Artificial sequence
  CCL20 smFISH probe 30
aacagtttgtgtgccgagaa

SEQ ID NO: 132 - DNA, 20 bp, Artificial sequence
  CCL20 smFISH probe 31
ggtcgttgagaaccaatcta

SEQ ID NO: 133 - DNA, 20 bp, Artificial sequence
  CCL20 smFISH probe 32
aatgtcttggaaacctttgt

SEQ ID NO: 134 - DNA, 20 bp, Artificial sequence
  CCL20 smFISH probe 33
aaccacttagaggaggagtt

SEQ ID NO: 135 - DNA, 20 bp, Artificial sequence
  CCL20 smFISH probe 34
acttatcctgtgattggtat

SEQ ID NO: 136 - DNA, 20 bp, Artificial sequence
  CCL20 smFISH probe 35
aggtattggatccacatgaa

SEQ ID NO: 137 - DNA, 20 bp, Artificial sequence
  CCL20 smFISH probe 36
gacagcagtcaaagttgctt

SEQ ID NO: 138 - DNA, 20 bp, Artificial sequence
  CCL20 smFISH probe 37
agaatacggtctgtgtatcc

SEQ ID NO: 139 - DNA, 20 bp, Artificial sequence
  CCL20 smFISH probe 38
cgtgtgaagcccacaataaa

SEQ ID NO: 140 - DNA, 20 bp, Artificial sequence
  CCL20 smFISH probe 39
gatagcattgatgtcacagc

SEQ ID NO: 141 - DNA, 20 bp, Artificial sequence
  CCL20 smFISH probe 40
agtttctaggtattcctgat

SEQ ID NO: 142 - DNA, 20 bp, Artificial sequence
  CCL20 smFISH probe 41
tttgccacatcatttctttc

SEQ ID NO: 143 - DNA, 20 bp, Artificial sequence
  CCL20 smFISH probe 42
aaaggttttgccttattcca

SEQ ID NO: 144 - DNA, 20 bp, Artificial sequence
  CCL20 smFISH probe 43
actagttccgtttgaccaaa

SEQ ID NO: 145 - DNA, 20 bp, Artificial sequence
  CCL20 smFISH probe 44
tagaaagtgatttttgccct

SEQ ID NO: 146 - DNA, 20 bp, Artificial sequence
  CCL20 smFISH probe 45
atcttccctattttcctaat

SEQ ID NO: 147 - DNA, 20 bp, Artificial sequence
  CCL20 smFISH probe 46
ctttctggtagaagatgctc

SEQ ID NO: 148 - DNA, 20 bp, Artificial sequence
  CCL20 smFISH probe 47
agattttacttttggtctca

SEQ ID NO: 149 - DNA, 20 bp, Artificial sequence
  CXCL2 smFISH probe 1
agggaatctcgtgagacagg

SEQ ID NO: 150 - DNA, 20 bp, Artificial sequence
  CXCL2 smFISH probe 2
cattcttgagtgtggctctg

SEQ ID NO: 151 - DNA, 20 bp, Artificial sequence
  CXCL2 smFISH probe 3
gggttgagacaagctttctg

SEQ ID NO: 152 - DNA, 20 bp, Artificial sequence
  CXCL2 smFISH probe 4
tttcttaaccatgggcgatg

SEQ ID NO: 153 - DNA, 20 bp, Artificial sequence
  CXCL2 smFISH probe 5
ctttttcagcatcttttcga

SEQ ID NO: 154 - DNA, 20 bp, Artificial sequence
  CXCL2 smFISH probe 6
cgcctgtgtacatggaaatt

SEQ ID NO: 155 - DNA, 20 bp, Artificial sequence
  CXCL2 smFISH probe 7
gtatttctgaccaacagctc

SEQ ID NO: 156 - DNA, 20 bp, Artificial sequence
  CXCL2 smFISH probe 8
actttttagggggcagacgc

SEQ ID NO: 157 - DNA, 20 bp, Artificial sequence
  CXCL2 smFISH probe 9
taaccctgggttttcctgat

SEQ ID NO: 158 - DNA, 20 bp, Artificial sequence
  CXCL2 smFISH probe 10
ataattttttcagtcctgca

SEQ ID NO: 159 - DNA, 20 bp, Artificial sequence
  CXCL2 smFISH probe 11
ccttaatggcaactttgtga

SEQ ID NO: 160 - DNA, 20 bp, Artificial sequence
  CXCL2 smFISH probe 12
tctggcaccagaacagatta

SEQ ID NO: 161 - DNA, 20 bp, Artificial sequence
  CXCL2 smFISH probe 13
aggagggaagaacctgggtg

SEQ ID NO: 162 - DNA, 20 bp, Artificial sequence
  CXCL2 smFISH probe 14
gcagttttacctacattcac

SEQ ID NO: 163 - DNA, 20 bp, Artificial sequence
  CXCL2 smFISH probe 15
tccaatgagaccttacatga

SEQ ID NO: 164 - DNA, 20 bp, Artificial sequence
  CXCL2 smFISH probe 16
cacttcagcacaactggcaa

SEQ ID NO: 165 - DNA, 20 bp, Artificial sequence
  CXCL2 smFISH probe 17
gtgttttttaacaggtggaa

SEQ ID NO: 166 - DNA, 20 bp, Artificial sequence
  CXCL2 smFISH probe 18
ggaactaggggaaatgaagc

SEQ ID NO: 167 - DNA, 20 bp, Artificial sequence
  CXCL2 smFISH probe 19
gcttttcaggtctgaaagtc

SEQ ID NO: 168 - DNA, 20 bp, Artificial sequence
  CXCL2 smFISH probe 20
tttacaaaaaaccagggggt

SEQ ID NO: 169 - DNA, 20 bp, Artificial sequence
  CXCL2 smFISH probe 21
attgctctttgtcatactgg

SEQ ID NO: 170 - DNA, 20 bp, Artificial sequence
  CXCL2 smFISH probe 22
ctggctaaaacagccagagc

SEQ ID NO: 171 - DNA, 20 bp, Artificial sequence
  CXCL2 smFISH probe 23
agtggcagtctacactgttt

SEQ ID NO: 172 - DNA, 20 bp, Artificial sequence
  CXCL2 smFISH probe 24
tcttttaggaatgggggtga

SEQ ID NO: 173 - DNA, 20 bp, Artificial sequence
  CXCL2 smFISH probe 25
tgtgtggcaaggacctctag

SEQ ID NO: 174 - DNA, 20 bp, Artificial sequence
  CXCL2 smFISH probe 26
aagtgggtactgcctgtgac

SEQ ID NO: 175 - DNA, 20 bp, Artificial sequence
  CXCL2 smFISH probe 27
gttttctgaatgcagtttcc

SEQ ID NO: 176 - DNA, 20 bp, Artificial sequence
  CXCL2 smFISH probe 28
cacaacagtcttctcctgtc

SEQ ID NO: 177 - DNA, 20 bp, Artificial sequence
  CXCL2 smFISH probe 29
taggtgctcaggaaagctga

SEQ ID NO: 178 - DNA, 20 bp, Artificial sequence
  CXCL2 smFISH probe 30
aatgagaaaatgggtgccct

SEQ ID NO: 179 - DNA, 20 bp, Artificial sequence
  CXCL2 smFISH probe 31
cttctggtcagttggatttg

SEQ ID NO: 180 - DNA, 20 bp, Artificial sequence
  CXCL2 smFISH probe 32
aacagccaccaataagcttc

SEQ ID NO: 181 - DNA, 20 bp, Artificial sequence
  CXCL2 smFISH probe 33
ctcttctgttcctgtaaggg

SEQ ID NO: 182 - DNA, 20 bp, Artificial sequence
  CXCL2 smFISH probe 34
aacacattaggcgcaatcca

SEQ ID NO: 183 - DNA, 20 bp, Artificial sequence
  CXCL2 smFISH probe 35
agacttctcctaagtgatgc

SEQ ID NO: 184 - DNA, 20 bp, Artificial sequence
  CXCL2 smFISH probe 36
gggagagtgtgcaagtagat

SEQ ID NO: 185 - DNA, 20 bp, Artificial sequence
  CXCL2 smFISH probe 37
actaacttgggtttgaccta

SEQ ID NO: 186 - DNA, 20 bp, Artificial sequence
  CXCL2 smFISH probe 38
ctgcaaaccttctatcttca

SEQ ID NO: 187 - DNA, 20 bp, Artificial sequence
  CXCL1 smFISH probe 1
tcactgactgagcgaggctg

SEQ ID NO: 188 - DNA, 20 bp, Artificial sequence
  CXCL1 smFISH probe 2
acgctcctagggaagaagag

SEQ ID NO: 189 - DNA, 20 bp, Artificial sequence
  CXCL1 smFISH probe 3
atgttcttggggtgaattcc

SEQ ID NO: 190 - DNA, 20 bp, Artificial sequence
  CXCL1 smFISH probe 4
ggacttcacgttcacacttt

SEQ ID NO: 191 - DNA, 20 bp, Artificial sequence
  CXCL1 smFISH probe 5
ggacttacatgacttcggtt

SEQ ID NO: 192 - DNA, 20 bp, Artificial sequence
  CXCL1 smFISH probe 6
agggaagggaatctcgtgag

SEQ ID NO: 193 - DNA, 20 bp, Artificial sequence
  CXCL1 smFISH probe 7
cattcttgagtgtggctctg

SEQ ID NO: 194 - DNA, 20 bp, Artificial sequence
  CXCL1 smFISH probe 8
caggattgaggcaagctttc

SEQ ID NO: 195 - DNA, 20 bp, Artificial sequence
  CXCL1 smFISH probe 9
attttcttaactatggggga

SEQ ID NO: 196 - DNA, 20 bp, Artificial sequence
  CXCL1 smFISH probe 10
taactcacctgttcagcatc

SEQ ID NO: 197 - DNA, 20 bp, Artificial sequence
  CXCL1 smFISH probe 11
tcgcctgtgtacatggaaac

SEQ ID NO: 198 - DNA, 20 bp, Artificial sequence
  CXCL1 smFISH probe 12
cagtatttctgaccaacggc

SEQ ID NO: 199 - DNA, 20 bp, Artificial sequence
  CXCL1 smFISH probe 13
ttttatttttagggggcaca

SEQ ID NO: 200 - DNA, 20 bp, Artificial sequence
  CXCL1 smFISH probe 14
caactaacccctgggttttc

SEQ ID NO: 201 - DNA, 20 bp, Artificial sequence
  CXCL1 smFISH probe 15
ataatcccaatttctagtcc

SEQ ID NO: 202 - DNA, 20 bp, Artificial sequence
  CXCL1 smFISH probe 16
gcaccagagcagtaattatc

SEQ ID NO: 203 - DNA, 20 bp, Artificial sequence
  CXCL1 smFISH probe 17
caggcattgggaatatcctc

SEQ ID NO: 204 - DNA, 20 bp, Artificial sequence
  CXCL1 smFISH probe 18
cgaaggaagaaccagggtgg

SEQ ID NO: 205 - DNA, 20 bp, Artificial sequence
  CXCL1 smFISH probe 19
gttttacctacattcattgg

SEQ ID NO: 206 - DNA, 20 bp, Artificial sequence
  CXCL1 smFISH probe 20
tactgggcctcaaatgaagg

SEQ ID NO: 207 - DNA, 20 bp, Artificial sequence
  CXCL1 smFISH probe 21
aaacagccagaacctgttgc

SEQ ID NO: 208 - DNA, 20 bp, Artificial sequence
  CXCL1 smFISH probe 22
tctccactgtactattggat

SEQ ID NO: 209 - DNA, 20 bp, Artificial sequence
  CXCL1 smFISH probe 23
cttttaggaatgggggtggg

SEQ ID NO: 210 - DNA, 20 bp, Artificial sequence
  CXCL1 smFISH probe 24
ggacctctaagcttgggatg

SEQ ID NO: 211 - DNA, 20 bp, Artificial sequence
  CXCL1 smFISH probe 25
tatgacagctgtgctgtgtg

SEQ ID NO: 212 - DNA, 20 bp, Artificial sequence
  CXCL1 smFISH probe 26
tggcaaccaagtggctactg

SEQ ID NO: 213 - DNA, 20 bp, Artificial sequence
  CXCL1 smFISH probe 27
agagttttccgaatgcagtt

SEQ ID NO: 214 - DNA, 20 bp, Artificial sequence
  CXCL1 smFISH probe 28
attgcacaacactcttctcc

SEQ ID NO: 215 - DNA, 20 bp, Artificial sequence
  CXCL1 smFISH probe 29
gagtaggtgctcgggaaagc

SEQ ID NO: 216 - DNA, 20 bp, Artificial sequence
  CXCL1 smFISH probe 30
gcaatgagaaaatgggtgcc

SEQ ID NO: 217 - DNA, 20 bp, Artificial sequence
  CXCL1 smFISH probe 31
cttctggtcagttggatttg

SEQ ID NO: 218 - DNA, 20 bp, Artificial sequence
  CXCL1 smFISH probe 32
ttcaggaacagccaccagtg

SEQ ID NO: 219 - DNA, 20 bp, Artificial sequence
  CXCL1 smFISH probe 33
ctcttctgttcctataaggg

SEQ ID NO: 220 - DNA, 20 bp, Artificial sequence
  CXCL1 smFISH probe 34
acacattaggcacaatccag

SEQ ID NO: 221 - DNA, 20 bp, Artificial sequence
  CXCL1 smFISH probe 35
agaagacttctcctaagcga

SEQ ID NO: 222 - DNA, 20 bp, Artificial sequence
  CXCL1 smFISH probe 36
cccttaattattttacacct

SEQ ID NO: 223 - DNA, 20 bp, Artificial sequence
  CXCL1 smFISH probe 37
ataggacagtgtgcaggtag

SEQ ID NO: 224 - DNA, 20 bp, Artificial sequence
  CXCL1 smFISH probe 38
acttggggttgacatttcaa

SEQ ID NO: 225 - DNA, 20 bp, Artificial sequence
  CXCL1 smFISH probe 39
gcaggctcctcagaaatatt

SEQ ID NO: 226 - DNA, 20 bp, Artificial sequence
  CXCL1 smFISH probe 40
ctatcacagtggctggcatg

SEQ ID NO: 227 - DNA, 20 bp, Artificial sequence
  CXCL1 smFISH probe 41
atgatctcattggccatttg

SEQ ID NO: 228 - DNA, 20 bp, Artificial sequence
  eGFP smFISH probe 1
tcctcgcccttgctcaccat

SEQ ID NO: 229 - DNA, 20 bp, Artificial sequence
  eGFP smFISH probe 2
atgggcaccaccccggtgaa

SEQ ID NO: 230 - DNA, 20 bp, Artificial sequence
  eGFP smFISH probe 3
ttacgtcgccgtccagctcg

SEQ ID NO: 231 - DNA, 20 bp, Artificial sequence
  eGFP smFISH probe 4
gacacgctgaacttgtggcc

SEQ ID NO: 232 - DNA, 20 bp, Artificial sequence
  eGFP smFISH probe 5
ggcatcgccctcgccctcgc

SEQ ID NO: 233 - DNA, 20 bp, Artificial sequence
  eGFP smFISH probe 6
tcagggtcagcttgccgtag

SEQ ID NO: 234 - DNA, 20 bp, Artificial sequence
  eGFP smFISH probe 7
ttgccggtggtgcagatgaa

SEQ ID NO: 235 - DNA, 20 bp, Artificial sequence
  eGFP smFISH probe 8
ggtgggccagggcacgggca

SEQ ID NO: 236 - DNA, 20 bp, Artificial sequence
  eGFP smFISH probe 9
cgtaggtgaaggtggtcacg

SEQ ID NO: 237 - DNA, 20 bp, Artificial sequence
  eGFP smFISH probe 10
tagcggctgaagcactgcac

SEQ ID NO: 238 - DNA, 20 bp, Artificial sequence
  eGFP smFISH probe 11
gtgctgcttcatgtggtcgg

SEQ ID NO: 239 - DNA, 20 bp, Artificial sequence
  eGFP smFISH probe 12
gcatggcggacttgaagaag

SEQ ID NO: 240 - DNA, 20 bp, Artificial sequence
  eGFP smFISH probe 13
cgctcctggacgtagccttc

SEQ ID NO: 241 - DNA, 20 bp, Artificial sequence
  eGFP smFISH probe 14
gtcgtccttgaagaagatgg

SEQ ID NO: 242 - DNA, 20 bp, Artificial sequence
  eGFP smFISH probe 15
cggcgcgggtcttgtagttg

SEQ ID NO: 243 - DNA, 20 bp, Artificial sequence
  eGFP smFISH probe 16
gtgtcgccctcgaacttcac

SEQ ID NO: 244 - DNA, 20 bp, Artificial sequence
  eGFP smFISH probe 17
ttcagctcgatgcggttcac

SEQ ID NO: 245 - DNA, 20 bp, Artificial sequence
  eGFP smFISH probe 18
cgtcctccttgaagtcgatg

SEQ ID NO: 246 - DNA, 20 bp, Artificial sequence
  eGFP smFISH probe 19
agcttgtgccccaggatgtt

SEQ ID NO: 247 - DNA, 20 bp, Artificial sequence
  eGFP smFISH probe 20
gtggctgttgtagttgtact

SEQ ID NO: 248 - DNA, 20 bp, Artificial sequence
  eGFP smFISH probe 21
tgtcggccatgatatagacg

SEQ ID NO: 249 - DNA, 20 bp, Artificial sequence
  eGFP smFISH probe 22
accttgatgccgttcttctg

SEQ ID NO: 250 - DNA, 20 bp, Artificial sequence
  eGFP smFISH probe 23
tgttgtggcggatcttgaag

SEQ ID NO: 251 - DNA, 20 bp, Artificial sequence
  eGFP smFISH probe 24
agctgcacgctgccgtcctc

SEQ ID NO: 252 - DNA, 20 bp, Artificial sequence
  eGFP smFISH probe 25
tgttctgctggtagtggtcg

SEQ ID NO: 253 - DNA, 20 bp, Artificial sequence
  eGFP smFISH probe 26
cacggggccgtcgccgatgg

SEQ ID NO: 254 - DNA, 20 bp, Artificial sequence
  eGFP smFISH probe 27
ctcaggtagtggttgtcggg

SEQ ID NO: 255 - DNA, 20 bp, Artificial sequence
  eGFP smFISH probe 28
ctttgctcagggcggactgg

SEQ ID NO: 256 - DNA, 20 bp, Artificial sequence
  eGFP smFISH probe 29
tgatcgcgcttctcgttggg

SEQ ID NO: 257 - DNA, 20 bp, Artificial sequence
  eGFP smFISH probe 30
cacgaactccagcaggacca

SEQ ID NO: 258 - DNA, 20 bp, Artificial sequence
  eGFP smFISH probe 31
cgagagtgatcccggcggcg

SEQ ID NO: 259 - DNA, 20 bp, Artificial sequence
  eGFP smFISH probe 32
cacttgtacagctcgtccat

SEQ ID NO: 260 - DNA, 20 bp, Artificial sequence
  UMLILO intron 1 smFISH probe 1
gctgcacatatatttatgtc

SEQ ID NO: 261 - DNA, 20 bp, Artificial sequence
  UMLILO intron 1 smFISH probe 2
gcctagaatgcttatgttgt

SEQ ID NO: 262 - DNA, 20 bp, Artificial sequence
  UMLILO intron 1 smFISH probe 3
ggacagtacacatccatttt

SEQ ID NO: 263 - DNA, 20 bp, Artificial sequence
  UMLILO intron 1 smFISH probe 4
atcttcctctttatacgggg

SEQ ID NO: 264 - DNA, 20 bp, Artificial sequence
  UMLILO intron 1 smFISH probe 5
aggtttcatcagtgtttgga

SEQ ID NO: 265 - DNA, 20 bp, Artificial sequence
  UMLILO intron 1 smFISH probe 6
gaactgcagagtatgttgtt

SEQ ID NO: 266 - DNA, 20 bp, Artificial sequence
  UMLILO intron 1 smFISH probe 7
attctaggtaggaacgatct

SEQ ID NO: 267 - DNA, 20 bp, Artificial sequence
  UMLILO intron 1 smFISH probe 8
agtcttgtttgcacattctt

SEQ ID NO: 268 - DNA, 20 bp, Artificial sequence
  UMLILO intron 1 smFISH probe 9
catatgcctcatttttccaa

SEQ ID NO: 269 - DNA, 20 bp, Artificial sequence
  UMLILO intron 1 smFISH probe 10
tgaagctcatgactgtgcat

SEQ ID NO: 270 - DNA, 20 bp, Artificial sequence
  UMLILO intron 1 smFISH probe 11
caagtgacttagtgttcccc

SEQ ID NO: 271 - DNA, 20 bp, Artificial sequence
  UMLILO intron 1 smFISH probe 12
accagagttcctttctcatg

SEQ ID NO: 272 - DNA, 20 bp, Artificial sequence
  UMLILO intron 1 smFISH probe 13
ctctgggaaaggacaggaca

SEQ ID NO: 273 - DNA, 20 bp, Artificial sequence
  UMLILO intron 1 smFISH probe 14
cttagaagtgttaggatccc

SEQ ID NO: 274 - DNA, 20 bp, Artificial sequence
  UMLILO intron 1 smFISH probe 15
aactagaggctaccaatggc

SEQ ID NO: 275 - DNA, 20 bp, Artificial sequence
  UMLILO intron 1 smFISH probe 16
ttagcggacaaacagttctg

SEQ ID NO: 276 - DNA, 20 bp, Artificial sequence
  UMLILO intron 1 smFISH probe 17
tcagcatcattttgctgtta

SEQ ID NO: 277 - DNA, 20 bp, Artificial sequence
  UMLILO intron 1 smFISH probe 18
tctcaacacacatcctgagc

SEQ ID NO: 278 - DNA, 20 bp, Artificial sequence
  UMLILO intron 1 smFISH probe 19
tgacaaaacccagttctgtc

SEQ ID NO: 279 - DNA, 20 bp, Artificial sequence
  UMLILO intron 1 smFISH probe 20
tgggtgtgaataacggcttg

SEQ ID NO: 280 - DNA, 20 bp, Artificial sequence
  UMLILO intron 1 smFISH probe 21
caaaccttggcctaggtgac

SEQ ID NO: 281 - DNA, 20 bp, Artificial sequence
  UMLILO intron 1 smFISH probe 22
cctcatttcacagttgaagt

SEQ ID NO: 282 - DNA, 20 bp, Artificial sequence
  UMLILO intron 1 smFISH probe 23
tttctatggcctcttgattt

SEQ ID NO: 283 - DNA, 20 bp, Artificial sequence
  UMLILO intron 1 smFISH probe 24
ctcatgcagtaatgctgtga

SEQ ID NO: 284 - DNA, 20 bp, Artificial sequence
  UMLILO intron 1 smFISH probe 25
gccagatattatgctgtttt

SEQ ID NO: 285 - DNA, 20 bp, Artificial sequence
  UMLILO intron 1 smFISH probe 26
aacaagctttacctgcagtg

SEQ ID NO: 286 - DNA, 20 bp, Artificial sequence
  UMLILO intron 1 smFISH probe 27
cattttgctttgagaagtcc

SEQ ID NO: 287 - DNA, 20 bp, Artificial sequence
  UMLILO intron 1 smFISH probe 28
gatgtcctgatattagcaga

SEQ ID NO: 288 - DNA, 20 bp, Artificial sequence
  UMLILO intron 1 smFISH probe 29
agagtctatcttaagctttt

SEQ ID NO: 289 - DNA, 20 bp, Artificial sequence
  UMLILO intron 1 smFISH probe 30
gattttctcattactacctt

SEQ ID NO: 290 - DNA, 20 bp, Artificial sequence
  UMLILO intron 1 smFISH probe 31
ctccttctagctattcttta

SEQ ID NO: 291 - DNA, 20 bp, Artificial sequence
  UMLILO intron 1 smFISH probe 32
ctgattttctcattattacc

SEQ ID NO: 292 - DNA, 20 bp, Artificial sequence
  UMLILO intron 1 smFISH probe 33
ccttctagctatcctttaat

SEQ ID NO: 293 - DNA, 20 bp, Artificial sequence
  UMLILO intron 1 smFISH probe 34
cttgttcttttcctttcatc

SEQ ID NO: 294 - DNA, 20 bp, Artificial sequence
  UMLILO intron 1 smFISH probe 35
gagcgtgtcacacagagtaa

SEQ ID NO: 295 - DNA, 20 bp, Artificial sequence
  UMLILO exon smFISH probe 1
ggtcttaatctccacatgta

SEQ ID NO: 296 - DNA, 20 bp, Artificial sequence
  UMLILO exon smFISH probe 2
gagtgttgctgcgagaattt

SEQ ID NO: 297 - DNA, 20 bp, Artificial sequence
  UMLILO exon smFISH probe 3
aagatccacacataaagcgc

SEQ ID NO: 298 - DNA, 20 bp, Artificial sequence
  UMLILO exon smFISH probe 4
gtagggttaatatgcagact

SEQ ID NO: 299 - DNA, 20 bp, Artificial sequence
  UMLILO exon smFISH probe 5
tgtttgagaggagccatttc

SEQ ID NO: 300 - DNA, 20 bp, Artificial sequence
  UMLILO exon smFISH probe 6
tacatttgggctgctctatc

SEQ ID NO: 301 - DNA, 20 bp, Artificial sequence
  UMLILO exon smFISH probe 7
tggaaggcaccacagtagca

SEQ ID NO: 302 - DNA, 20 bp, Artificial sequence
  UMLILO exon smFISH probe 8
tcctcagagagttttgcatt

SEQ ID NO: 303 - DNA, 20 bp, Artificial sequence
  UMLILO exon smFISH probe 9
ggtagcagtagacatattct

SEQ ID NO: 304 - DNA, 20 bp, Artificial sequence
  UMLILO exon smFISH probe 10
actagatgatgggggttaca

SEQ ID NO: 305 - DNA, 20 bp, Artificial sequence
  UMLILO exon smFISH probe 11
aatgttcaccgtgtaccaac

SEQ ID NO: 306 - DNA, 20 bp, Artificial sequence
  UMLILO exon smFISH probe 12
agcagaatacattcccacag

SEQ ID NO: 307 - DNA, 20 bp, Artificial sequence
  UMLILO exon smFISH probe 13
aattggcaggcctctggaat

SEQ ID NO: 308 - DNA, 20 bp, Artificial sequence
  UMLILO exon smFISH probe 14
ttcagcctatagtgagatta

SEQ ID NO: 309 - DNA, 20 bp, Artificial sequence
  UMLILO exon smFISH probe 15
aagtattctatgtgagcagc

SEQ ID NO: 310 - DNA, 20 bp, Artificial sequence
  UMLILO exon smFISH probe 16
gtccactgatctaaactggt

SEQ ID NO: 311 - DNA, 20 bp, Artificial sequence
  UMLILO exon smFISH probe 17
agcagtaattcagcatctct

SEQ ID NO: 312 - DNA, 20 bp, Artificial sequence
  UMLILO exon smFISH probe 18
ggcatgtggatctatacttc

SEQ ID NO: 313 - DNA, 20 bp, Artificial sequence
  UMLILO exon smFISH probe 19
actcaacattcgcctctaat

SEQ ID NO: 314 - DNA, 20 bp, Artificial sequence
  UMLILO exon smFISH probe 20
gataaatacagttcctgcct